(19) United States
(12) Patent Application Publication
(10) Pub. No.: US 2012/0237532 A1
(43) Pub. Date: Sep. 20, 2012

(54) PHARMACEUTICAL FORMULATION CONTAINING IMMUNOGLOBULIN

(75) Inventors: Carsten Olbrich, Berlin (DE); Michael Krause, Berlin (DE); Thomas Trill, Schildow (DE)

(73) Assignee: Icon Genetics GmbH, Munich (DE)

(21) Appl. No.: 13/415,098

(22) Filed: Mar. 8, 2012

(30) Foreign Application Priority Data
Mar. 15, 2011 (EP) 11 002 145.8

(51) Int. Cl. A61K 39/44; A61P 35/04

(52) U.S. Cl. 424/178.1

(57) ABSTRACT

A set of at least two different protein conjugate preparations, each protein conjugate preparation comprising histidine as a buffering agent and a protein conjugate comprising one or more immunoglobulin moieties conjugated to a carrier protein; wherein the immunoglobulin moieties of each element of said set of protein conjugate preparation have identical complementarity determining regions (CDRs); and wherein different protein conjugate preparations differ in that the immunoglobulin moieties of the protein conjugates have different CDRs.

### CROSS-REFERENCE TO RELATED APPLICATIONS

The present patent application claims the benefit of priority from European patent application No. 11002145.8, filed on Mar. 15, 2011, the entire content of which is incorporated herein by reference.

### FIELD OF THE INVENTION

The present invention relates to a pharmaceutical preparation and an aqueous formulation comprising an immunoglobulin or a protein conjugate comprising an immunoglobulin conjugated to a carrier protein. The pharmaceutical formulation may be for parenteral administration such as subcutaneous administration for the treatment of B-cell non-Hodgkin lymphoma. The invention further relates to a set of at least two different protein conjugate preparations.

### BACKGROUND OF THE INVENTION

Anti-idiotype antibodies and vaccines are a promising tool for the treatment of various cancers such as B-cell lymphoma, see López-Diaz de Cerio et al., Oncogene 26 (2007) 3594-3602; Levy, J. Clinical Oncology 17(11s) (1999) 7-13; J Natl Cancer Inst 98 (2006) 1292-1301. A blood cancer for which successful treatment has been demonstrated in clinical experiments is non-Hodgkin lymphoma (herein: NHL). Tumor therapy by anti-idiotype antibodies or vaccines requires patient-specific antibodies or vaccines, respectively, which represents a major challenge for large-scale application to many patients, since sufficient amounts of antibody or vaccine have to be produced for each patient separately within a reasonable time and at reasonable costs. Several technologies for the production of human immunoglobulins are known such as the concomitant production of heavy and light chains of immunoglobulin G in plants (WO2006/079546; Giritch et al., Proc. Natl. Acad. Sci. USA 2006 103 (40) 14701-14706; McCormick et al., Proc. Natl. Acad. Sci. USA 96 (1999) 703-708; Osterroth et al., Journal of Immunological Methods 229 (1999) 141-153). The technical challenges for cloning of relevant complementarity determining regions (CDRs) from autopsy-derived RNA and expression of heavy and light chains of immunoglobulins is also feasible. Due to the enormous costs for providing patient-specific immunoglobulins, automation of as many production steps as possible is imperative.

A problem inherent to the production of patient-specific medicaments is that the physical properties of individual immunoglobulins vary due to the differences in their variable regions. These differences affect the solubility and stability of the immunoglobulin preparations. It has been observed that immunoglobulins differing in their variable region have a high variability in terms of solubility of the antibodies in typical buffers used for extracting the immunoglobulins from cells as well as buffers used for downstream processing. In order to be able to provide predetermined dosages of the immunoglobulin to the medical personnel for administration to patients, immunoglobulin preparations must fulfil certain minimum stability requirements. However, it is not possible in a standardized manufacturing procedure to search for optimal conditions for each individual immunoglobulin. Thus, conditions must be found that allow achieving sufficiently stable immunoglobulin preparations over a wide range of immunoglobulins differing in their variable region.

It is therefore an object of the present invention to provide a pharmaceutical formulation of immunoglobulins, notably of IgG, and of their conjugates with suitable carrier proteins that provides high stability and solubility of the immunoglobulins and their conjugates. It is also an object to provide a method of treatment of B-cell lymphoma by anti-idiotype vaccines using the formulation of the present invention.

### SUMMARY OF THE INVENTION

The present invention provides a pharmaceutical preparation comprising a protein conjugate comprising an immunoglobulin (such as immunoglobulin G) conjugated to a carrier protein, and histidine as a buffering agent.

The present invention further provides an aqueous pharmaceutical formulation comprising a protein conjugate comprising an immunoglobulin (such as immunoglobulin G) conjugated to a carrier protein, and histidine as a buffering agent.

Further, the present invention provides a formulation for use in a therapeutic method such as for the treatment of non-Hodgkin lymphoma. The present invention provides a formulation for use in a method for the treatment of B-cell lymphoma, notably follicular B-cell non-Hodgkin lymphoma.

The present invention further provides a set of at least two different protein conjugate preparations, each protein conjugate preparation comprising histidine as a buffering agent and a protein conjugate comprising one or more immunoglobulin moieties conjugated to a carrier protein; wherein the immunoglobulin moieties of each protein conjugate preparation have identical complementarity determining regions (CDRs); and wherein different protein conjugate preparations differ in that the immunoglobulin moieties of the protein conjugates have different CDRs. In one embodiment, the immunoglobulin moieties of each protein conjugate preparation have identical variable regions, and different protein conjugate preparations differ in that the immunoglobulin moieties of the protein conjugates have different variable regions.

Also provided is a method of treating a patient suffering from B-cell non-Hodgkin lymphoma (notably follicular B-cell lymphoma), comprising administering to the patient an aqueous pharmaceutical formulation, said formulation comprising a protein conjugate comprising an immunoglobulin conjugated to a carrier protein, and histidine as a buffering agent; wherein the complementarity determining regions of the immunoglobulin conjugated to the carrier protein elicits an immune response in the patient against the non-Hodgkin lymphoma B-cells.

In an important embodiment, the immunoglobulins used in the invention are plant-expressed immunoglobulins, such as plant-expressed IgGs. A preferred plant for expressing are Nicotiana plants such as Nicotiana benthamiana or Nicotiana tabacum.

Immunoglobulins and protein conjugates comprising immunoglobulins can vary considerably in their solubility and stability in solution due to differences in their variable region, notably in the complementary determining regions (CDR) of the immunoglobulins. It has now surprisingly been found by the inventors that aqueous formulations containing histidine as a buffering agent can stabilise a wide range of immunoglobulins and protein conjugates comprising immunoglobulins and a carrier protein. Using histidine as a buffering agent thus allows high throughput generation of different immunoglobulins and protein conjugates of sufficient stability without the need to search for suitable additives or buffers for immunoglobulins or protein conjugates of problematic stability. Further, use of histidine buffer, optionally in combination with suitable surfactants as described below, provides sufficient stability against shaking or shear stress as occurs during sterile filtration, storage, shipment or filtering of formulations before administration to patients. Thus, the invention provides an important contribution for making individualised medicine using immunoglobulins or protein conjugates thereof commercially viable.

### BRIEF DESCRIPTION OF THE FIGURES

FIG. 1 is a scheme showing amplification of variable region fragments of an immunoglobulin and cloning in viral vectors for heavy chain expression. BAP stands for an adaptor sequence as in FIG. 2 of WO 2010/040531. Nucleotide quadruplett AGGT is the cleavage site of the BsaI sites and, accordingly, the sequence of a single-stranded overhang produced by BsaI restriction. Similarly, NNNN stands for any predetermined sequence forming a single-stranded overhang produced by BsaI restriction. Rice ats is the rice amylase transit signal peptide.

FIG. 2 is a workflow showing steps for amplification, cloning, and expression in Nicotiana benthamiana of heavy and light IgG chains. See section “Preparation of idiotypic antibodies for vaccination against NHL” for more information on the working steps depicted.

FIGS. 3A and B show schematically binary vectors encoding RNA viral vectors used for expressing the light and heavy chains of Ig T038 used in the examples.

FIG. 3A depicts binary TMV-based vector pICOT-RL038 encoding the T038 light chain. RB and LB are the right and left T-DNA borders. Act2 indicates the rice actin2 promoter. The long extended box downstream from Act2 is a replicase ORF derived from tobacco mosaic virus (TMV); small boxes therein indicate artificially introduced introns. T038 VL2 is the light chain variable region of Ig T038 that is followed by the codon-optimised constant region of a human lambda chain. NTR stands for non-translated region. NOS stands for the nopaline synthase terminator. The amino acid sequence (SEQ ID NO: 3) and nucleotide sequence (SEQ ID NO: 4) of the fragment consisting of or encoding, respectively, the rice ats (shown in the first line of the sequences), the variable region, and the lambda constant region are shown.

FIG. 3B depicts binary vector PICOP-RH038 containing the T038 heavy chain. The potato virus X (PVX)-based vector is under the control of the 35S promoter. PVX Pol is the PVX polymerase. Downstream thereof, the subgenomic promoter sg25 is depicted followed by the PVX-based triple gene block encoding the 25K, 12K and 8K proteins. sgp is the PVX coat protein (CP) subgenomic promoter. T038 VH4 is the sequence encoding the variable heavy chain fragment of T038 linked to the human IgG1 constant region. CP3′ stands for the coat protein ORF located 3′ of the vector. The amino acid sequence (SEQ ID NO: 5) and nucleotide sequence (SEQ ID NO: 6) of the fragment consisting of and encoding, respectively, the rice ats (first line of the sequences shown), the variable region, and the IgG1 constant region are shown.

FIG. 4A shows schematically the binary PVX-based vector pICOP-BK069 encoding an RNA viral vector used for expressing the light chains of Ig T069 used in the examples. The amino acid sequence (SEQ ID NO: 7) of the fragment consisting the barley amylase ats (first line of the sequence), the variable region, and the kappa constant region is shown. The nucleotide sequence (SEQ ID NO: 8) of the fragment encoding the variable region and the kappa constant region is shown in the box at the bottom.

FIG. 4B shows at the top the TMV-based vector pICOT-BH069 used for expressing the heavy chain of Ig T069. The top box shows the amino acid sequence (SEQ ID NO: 9) of the fragment consisting of the barley amylase ats (first line), the variable domain, and the human IgG1 constant region of the T069 heavy chain. The box at the bottom shows the nucleotide sequence (SEQ ID NO: 10) of the fragment encoding the variable domain and the human IgG1 constant region of the T069 heavy chain.

FIG. 5A shows the PVX-based binary vector pICOP-BK096 usable for expressing the light chain of T096 and the amino acid sequence (SEQ ID NO: 11) and nucleotide sequence (SEQ ID NO: 12) of the fragment consisting of or encoding, respectively, the barley amylase ats (first lines of the sequences), the variable domain, and the kappa constant region of the T096 light chain.

FIG. 5B shows the TMV-based binary vector pICOT-BH096 usable for expressing the heavy chain of T096 and the amino acid sequence (SEQ ID NO: 13) and in the box at the bottom the nucleotide sequence (SEQ ID NO: 14) of the fragment consisting of or encoding, respectively, the barley ats, the variable domain, and the constant region of the T096 heavy chain.

FIG. 6A shows the amino acid sequence (SEQ ID NO: 15) of the fragment consisting of the barley apoplast leader (first line of the amino acid sequence), the variable domain, and the constant region of the T113 kappa light chain. Also shown is the nucleotide sequence (SEQ ID NO: 16) of the fragment encoding the variable domain and the constant region of the T113 kappa light chain.

FIG. 6B shows the amino acid sequence (SEQ ID NO: 17) of the fragment consisting of the barley ats (first line of the amino acid sequence), the variable domain, and the constant region of the T113 heavy chain of IgG1 T113. Also shown is the nucleotide sequence (SEQ ID NO: 18) of the fragment encoding the variable domain and the constant region of the T113 heavy chain of IgG1 T113.

FIG. 7A depicts the TMV-based binary vector PICOT-RL117 encoding the T117 light chain. The amino acid sequence (SEQ ID NO: 19) and the nucleotide sequence (SEQ ID NO: 20) of the fragment consisting of or encoding, respectively, the rice apoplast leader, the variable domain, and a human lambda constant region are shown.

FIG. 7B depicts binary vector PICOP-RH117 containing the T117 heavy chain with rice apoplast leader. The amino acid sequence (SEQ ID NO: 21) and the nucleotide sequence (SEQ ID NO: 22) of the fragment consisting of or encoding, respectively, the rice apoplast leader, the variable domain, and the IgG1 constant region are shown.

FIG. 8A depicts the PVX-based binary vector PICOP-RL118 encoding the T118 light chain. The amino acid sequence (SEQ ID NO: 23) and the nucleotide sequence (SEQ ID NO: 24) of the fragment consisting of or encoding, respectively, the rice apoplast leader (first line of the sequences), the variable domain, and a human lambda constant region are shown.

FIG. 8B depicts binary TMV-based vector PICOT-RH118 containing the T118 heavy chain with rice apoplast leader. The amino acid sequence (SEQ ID NO: 25) and the nucleotide sequence (SEQ ID NO: 26) of the fragment consisting of or encoding, respectively, the rice apoplast leader, the variable domain, and the IgG1 constant region are shown.

### DETAILED DESCRIPTION OF THE INVENTION

The immunoglobulin used in the invention may be an immunoglobulin A, M or G. Immunoglobulins A and G are preferred and immunoglobulin G is most preferred. Immunoglobulin G refers to immunoglobulin G molecules of the subclasses such as IgG1, IgG2, IgG3 or IgG4. Preferably, the immunoglobulin G of the present invention is of the subclass IgG1 or IgG3, more preferably IgG1.

Examples of immunoglobulins (Igs) to be used in the present invention are tumor-derived idiotypic immunoglobulins from non-Hodgkin lymphoma patients. For example, immunoglobulins G (IgGs) can be used for this purpose. Tumor-specific antibodies from non-Hodgkin lymphoma patients contain a collection of epitopes present in the variable region of the clonal immunoglobulin which can be used as a target for active immunotherapy. The tumor-specific immunoglobulins from individual B cell lymphomas can be identified and isolated using “rescue hybridization” or molecular cloning techniques (Levy and Dilley, PNAS, 75 (1978) 2411-2415; Hawkins et al., Blood, 83 (1994), 3279-3288), yielding patient-specific (idiotypic) immunoglobulin which can be used as a tumor antigen for vaccination. Immunoglobulins can also be recombinantly expressed in host cells. A suitable process of expressing immunoglobulins, including patient-specific idiotypic immunoglobulins, is expression in plants or plant cells as described in WO 2006/079546 and Giritch et al., Proc. Natl. Acad. Sci. USA 2006 103 (40) 14701-14706.

When used for vaccination such as in the treatment of non-Hodgkin lymphoma (NHL), the immunoglobulins are preferably administered together with suitable adjuvants for increasing immune response. Established adjuvants can be used for this purpose such as hGM-CSF (human granulocyte-macrophage colony stimulating factor). Another possibility preferred herein is conjugation of the immunoglobulins to a highly immunogenic carrier protein for enhancing the immunogenicity. It is also possible to combine various adjuvants. For example, a protein conjugate having the immunoglobulin conjugated to a carrier protein can be administered together with an adjuvant such as hGM-CSF.

Carrier proteins to which immunoglobulins can be conjugated are known to the skilled person. Examples are diphtheria toxin, tetanus toxin, human serum albumin (HSA), bovine serum albumin (BSA), or hemocyanin such as keyhole limpet hemocyanin (KLH) or subunits thereof. In one embodiment, BSA, hemocyanin (such as KLH) or subunits of hemocyanin are used as the carrier protein. In a preferred embodiment, hemocyanin (such as KLH) or a subunit thereof is used as carrier proteins. KLH is commercially available e.g. from biosyn GmbH (see below).

Hemocyanins are generally isolated from mollusks such as the giant keyhole limpet. Hemocyanins are oligomeric proteins i.e. have multiple subunits. The hemocyanin subunits associate to form the multi-subunit oligomers. Native KLH is a cylindrical copper containing blue protein with a molecular mass ranging from 8 to 32 MDa. The most prevalent oligomeric forms are didecamers with a molecular mass of 8 MDa and multidecamers with a molecular mass of 12-32 MDa. The didecamer has 20 subunits and occurs in two different forms named KLH1 and KLH2. The amino acid sequences of KLH1 and KLH2 are given in the annex as SEQ ID NO: 1 and SEQ ID NO:2, respectively. Instead of native KLH, subunits of KLH can be used as carrier protein in the present invention. Subunits of KLH are also referred to as immunocyanin. A commercially available immunocyanin is VACMUNE® from biosyn.

Herein, “protein conjugate” means a protein comprising one or more immunoglobulin molecules that are covalently bound to a carrier protein. Thus, a protein conjugate molecule comprises a carrier protein and one or more immunoglobulin moieties. The “carrier protein” may be an oligomer, as described above for hemocyanin. It is possible that the immunoglobulin moieties are bound to the carrier protein via linkers. If more than one immunoglobulin molecule is bound to a carrier protein, the immunoglobulin moieties generally have the same CDRs or the same variable regions. In one embodiment, the immunoglobulin moieties are identical.

Methods for conjugating proteins are known to the skilled person and are widely used for conjugating haptens to carrier proteins in processes of antibody generation, and suitable kits are commercially available. As an example for the conjugation reaction, the reaction of an immunoglobulin (such as an IgG) and the carrier protein using the bifunctional linker glutaraldehyde may be used.

In the conjugation reaction, the Ig (such as IgG) molecule and the carrier protein can be employed in different ratios depending on whether multiple immunoglobulin molecules are to be bound to a carrier protein. Accordingly, the molar ratio of Ig to carrier protein subunit may be from about 4:1 to about 1:1, preferably the molar ratio is from about 2:1 to 1:1. In terms of mass ratio, the mass ratio of Ig to carrier protein may be from about 1.5 to 0.4, preferably from about 0.75 to 0.4.

A commercial kit for conjugating proteins to KLH as a carrier protein and that has been used in the examples of this invention for conjugating idiotypic IgG molecules to a carrier protein is VACMUNE® (biosyn GmbH, Fellbach, Germany). In this system, proteins are conjugated to 400 kDa subunits of hemocyanin in water, using 0.1% glutaraldehyde as the conjugating reagent with the molar ratio of IgG to the 400 kDa KLH subunits of about 2.5:1 in the conjugation reaction. The resulting protein conjugate was further purified and can be formulated into the formulation of the present invention by introducing the reaction product in the desired buffer.

The preparations of the invention contain histidine as the buffering agent and the immunoglobulin or the protein conjugate comprising the immunoglobulin. The preparations of the invention may be aqueous liquid preparations or solid preparations. The solid preparations may be prepared from the aqueous liquid preparation by freeze-drying (lyophilisation). The lyophilised solid preparation may be reconstituted to form a liquid aqueous preparation by adding water or an aqueous solvent. An example of the aqueous liquid preparation is the aqueous pharmaceutical formulation of the invention that may, in addition to the immunoglobulin or the protein conjugate, histidine and water, further contain pharmaceutical excipients such as an isotonizing agent, a preservative or others (see further below).

In the aqueous formulation of the invention, the protein conjugate may be present in a concentration of from about 0.1 to about 10 mg/ml, preferably in an amount of from about 0.5 to about 5 mg/ml and more preferably of about 0.7 to 2.5 mg/ml.

The histidine concentration in the aqueous formulation or the aqueous liquid preparations may be 5 mM to 100 mM histidine, preferably 10 mM to 50 mM, more in one embodiment 20 mM to 30 mM histidine. Regarding the pH of the aqueous formulation or the aqueous liquid preparations, a pH which is close to the physiological pH of humans should be chosen when used for administration to patients. The pH may be between 7.0 and 7.8. In one embodiment it is between 7.2 and 7.6 and more preferably 7.3 to 7.5. The pH may be set as commonly known by adding an acid to an aqueous histidine solution. Examples of the acid are inorganic acids such as hydrochloric acid and sulfuric acid, or organic acids such as citric acid, lactic acid, tartaric acid and other physiologically acceptable acids.

The aqueous formulation or the aqueous liquid preparation of the present invention may optionally further contain a nonionic surfactant. Examples of surfactants are known to the skilled person e.g. from McCutcheon's Emulsifiers and Detergents, 1986 North American Edition, McCutcheon Division Publishing Co., Glen Rock, N.J. For medical applications such as for the formulation of the invention, pharmaceutically acceptable nonionic surfactants can be used as known by the skilled person. Combinations of nonionic surfactants can also be used in the present invention.

Among nonionic surfactants, polysorbate surfactants are particularly preferable. Polysorbate surfactants are understood herein to comprise polyoxyethylene sorbitan fatty acid esters, such as polysorbate 80 (poly(oxyethylene)sorbitan monooleate, also known as Tween-80), polysorbate 60 (poly(oxyethylene)sorbitan monostearate, also known as Tween-60), polysorbate 40 (poly(oxyethylene)sorbitan monopalmitate, also known as Tween-40), polysorbate 20 (poly(oxyethylene)sorbitan monolaurate, also known as Tween-20), polysorbate 85 (poly(oxyethylene)sorbitan trioleate, also known as Tween-85) and polysorbate 65 (poly(oxyethylene)sorbitan tristearate, also known as Tween-65). Of these, polysorbate 20 and polysorbate 80 are preferred, with polysorbate 80 being particularly preferred.

Other nonionic surfactants usable in the invention are poly(propylenoxide) and block copolymers of poly(propylenoxide) and poly(ethylenoxide) such as Synperonic® F-68, Pluronic® F68, Lutrol® F68 or Poloxamer 188.

Further additives to the formulations of the invention that may be used instead of the nonionic surfactants are cyclodextrins, such as HP-β-CD, that may be used for protein stabilisation and can be used for parenteral administration of drugs.

The non-ionic surfactant may be present in the aqueous formulation or the aqueous liquid preparation in a concentration of from about 0.001% to about 2% (w/v), preferably from about 0.01% to about 0.5% (w/v). In a preferred embodiment, the surfactant is present in a concentration of about 0.1% (w/v), in an alternative preferred embodiment in a concentration of about 0.01% (w/v).

The formulation or the liquid preparation of the present invention may comprise an isotonization agent. For the parenteral administration of a pharmaceutical formulation, it is desirable to adjust the tonicity of the formulation to the fluids of the human body. The isotonization agents to be used in the formulation of the present invention will be apparent to a person skilled in the art. However, sugar alcohols, polyvinyl pyrrolidone and disaccharides are explicitly mentioned. Of these, mannitol and disaccharides are preferred with disaccharides being more preferred. Particularly preferred disaccharides comprise trehalose and sucrose. In a preferred embodiment of the formulation of the present invention, trehalose is used as isotonization agent.

The pharmaceutical formulation of the invention may further contain one or more additional pharmaceutically acceptable excipients such as buffers, anti-oxidants, bacteriostatic agents, diluents, carriers, surfactants, salts, preserving, stabilizing, wetting or solubilizing agents, and/or excipients for regulating the osmolarity.

In one embodiment of the formulation of the present invention, the formulation comprises histidine as a buffering agent in an amount of from 10 mM to 50 mM and polysorbate 20 or polysorbate 80 in an amount of from about 0.01% to about 0.1% (w/v), and an isotonization agent. In another embodiment, the formulation comprises histidine as a buffering agent in an amount of from 10 mM to 50 mM and polysorbate 20 or polysorbate 80 in an amount of from about 0.01% to about 0.1% (w/v), an isotonization agent, and a protein conjugate comprising an immunoglobulin G conjugated to a carrier protein (e.g. KLH or a subunit thereof) in a concentration of from 0.1 to 10 mg/ml, the formulation having a pH of from 7.3 to 7.5.

Generally, a pharmaceutical formulation herein means a formulation which is suitable for administration to patients, notably of human patients. The pharmaceutical formulation may be administered to the patient by way of parenteral administration, preferably by subcutaneous or intramuscular administration, more preferably by way of subcutaneous administration.

The invention has its full potential when a set of at least two different protein conjugate preparations are to be prepared, such as for the production of multiple B-cell lymphoma vaccines for two or more patients. Use of a histidine buffer as described herein then allows to obtain two or more conjugate preparations, each having a high likelihood of having sufficient stability and solubility. Each of the multiple protein conjugate preparations comprises histidine as a buffering agent and a protein conjugate comprising one or more immunoglobulin moieties conjugated to a carrier protein. The immunoglobulin moieties of each element of said set of protein conjugate preparation have identical complementarity determining regions (CDRs) or identical variable regions. Different protein conjugate preparations of said set differ in that the immunoglobulin moieties of the protein conjugates have different CDRs and thus different variable regions.

A method of preparing a plurality of at least two different protein conjugate preparations as defined above may comprise the following steps:
(i) separately providing at least two different immunoglobulins;
(ii) separately conjugating the at least two different immunoglobulins to a carrier protein to obtain said plurality of at least two different protein conjugates;
(iii) separately introducing each protein conjugates of step (ii) into a histidine buffer to obtain the plurality of at least two different protein conjugate preparations.

The invention also provides a pharmaceutical preparation comprising an immunoglobulin and histidine as a buffering agent, and an aqueous pharmaceutical formulation comprising an immunoglobulin (such as immunoglobulin G) and histidine as a buffering agent.

Also provided is a set of at least two different immunoglobulin preparations, each immunoglobulin preparation comprising histidine as a buffering agent and an immunoglobulin; wherein the immunoglobulin molecules of each immunoglobulin preparation have identical CDRs; and wherein different immunoglobulin preparations differ in that the immunoglobulins of different preparations have different CDRs.

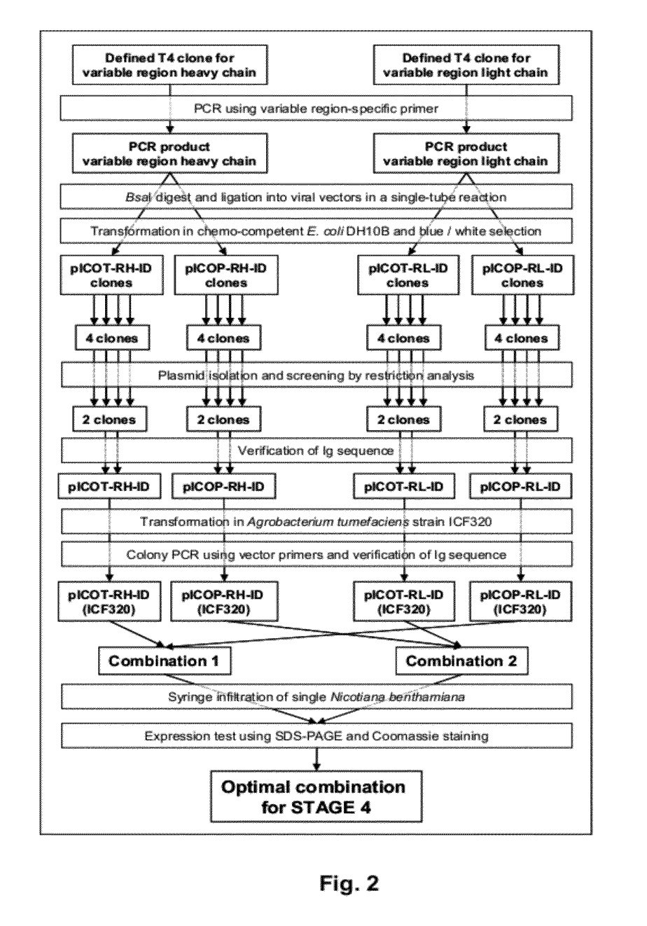

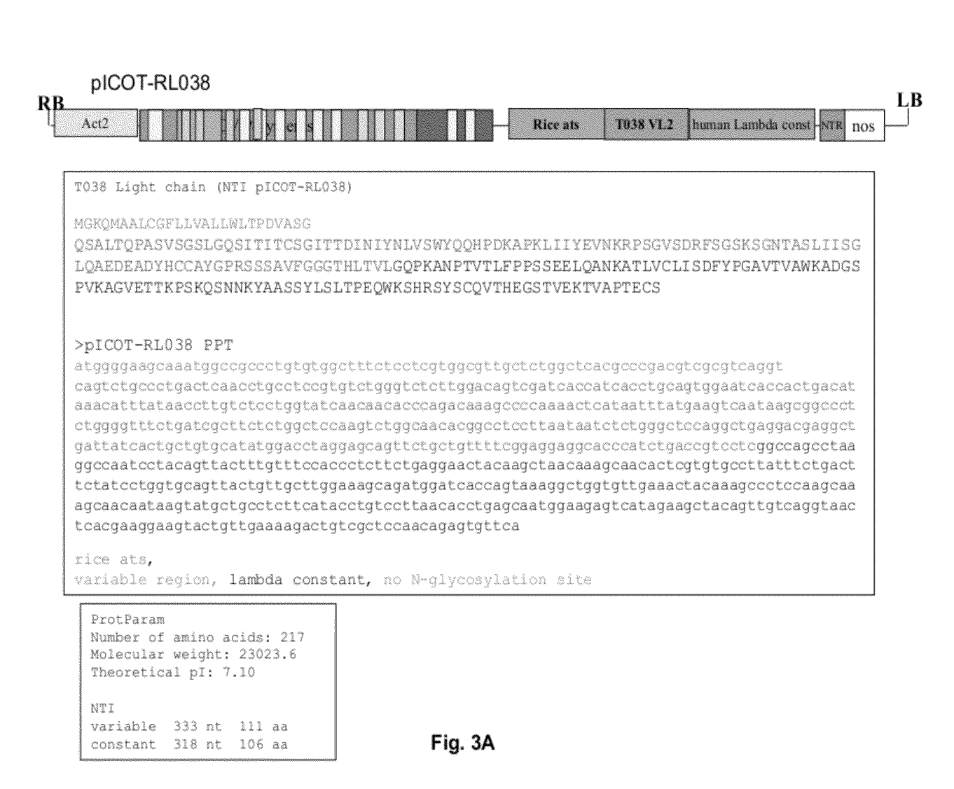

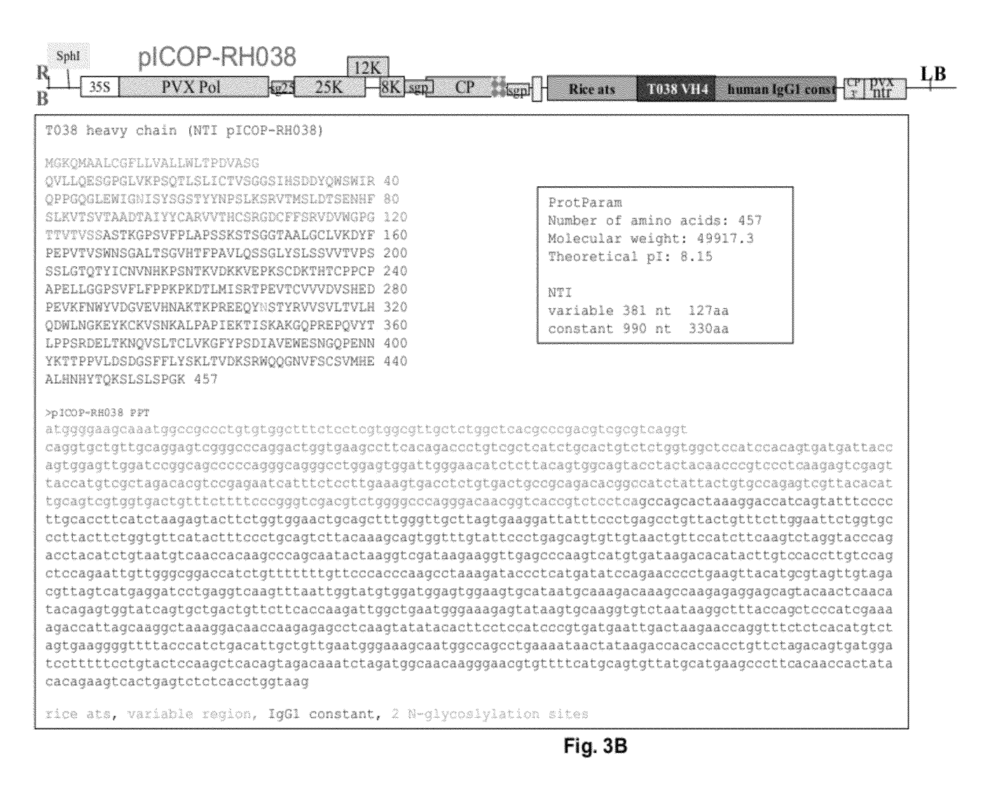

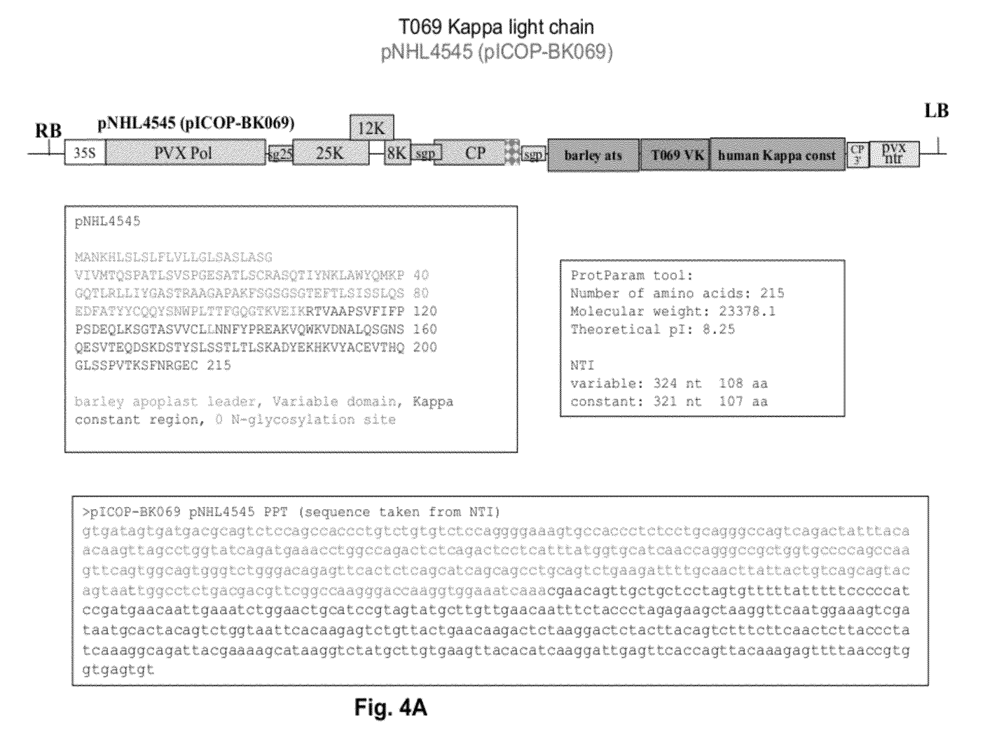

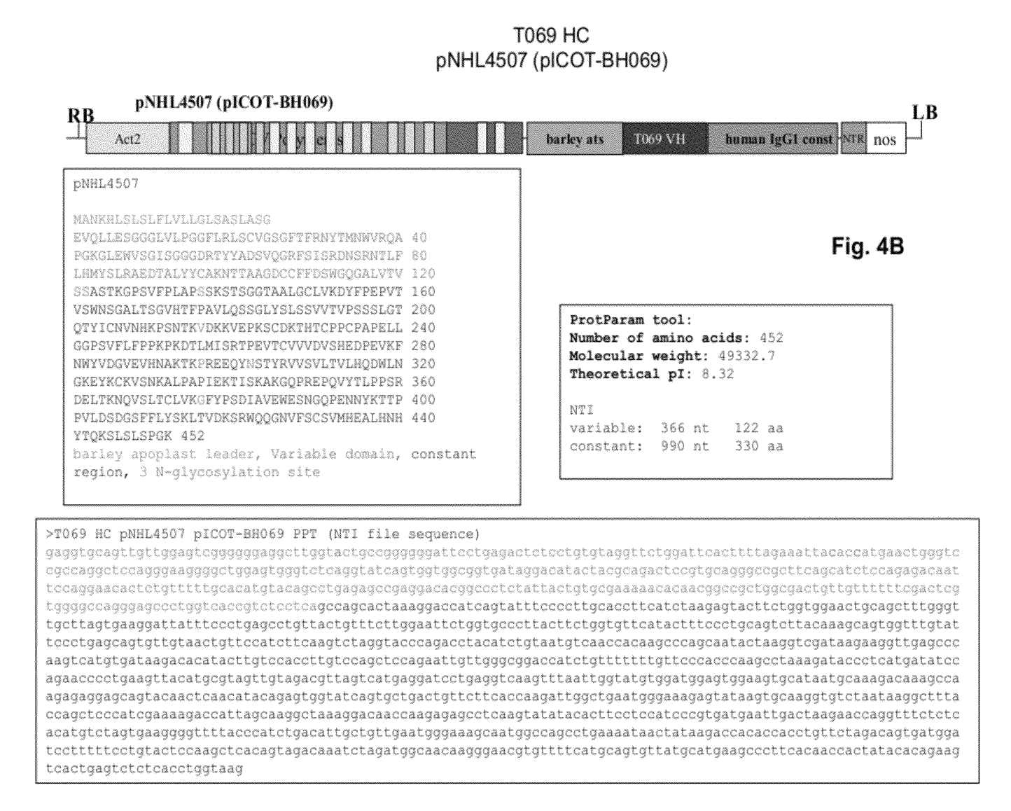

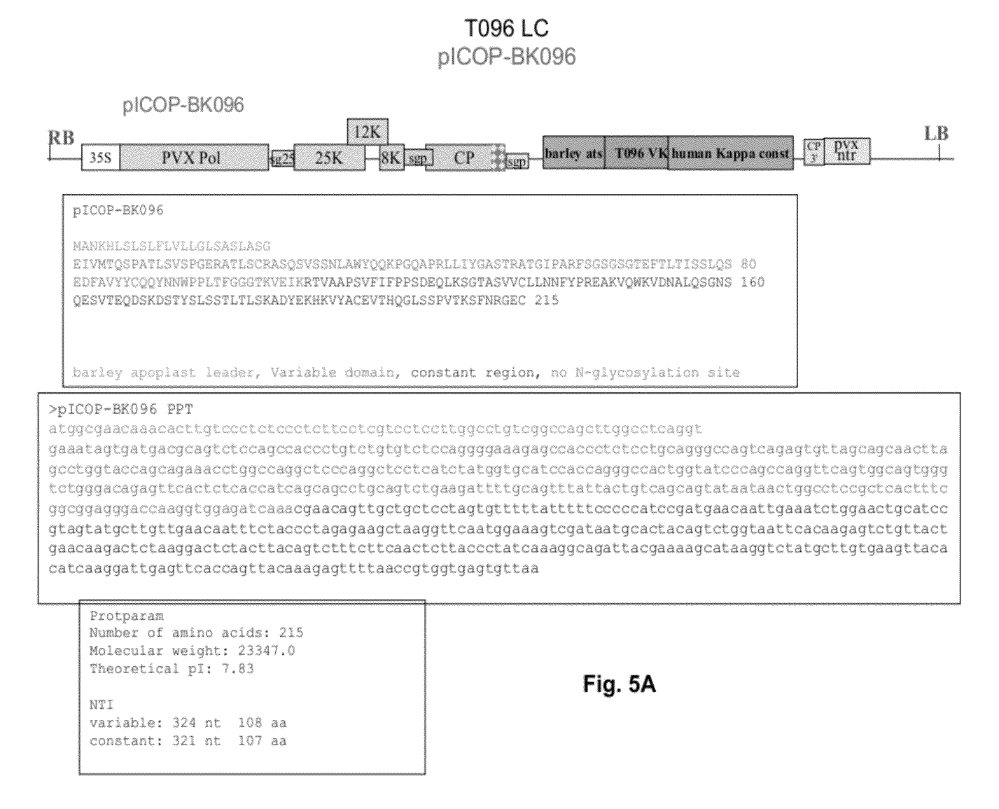

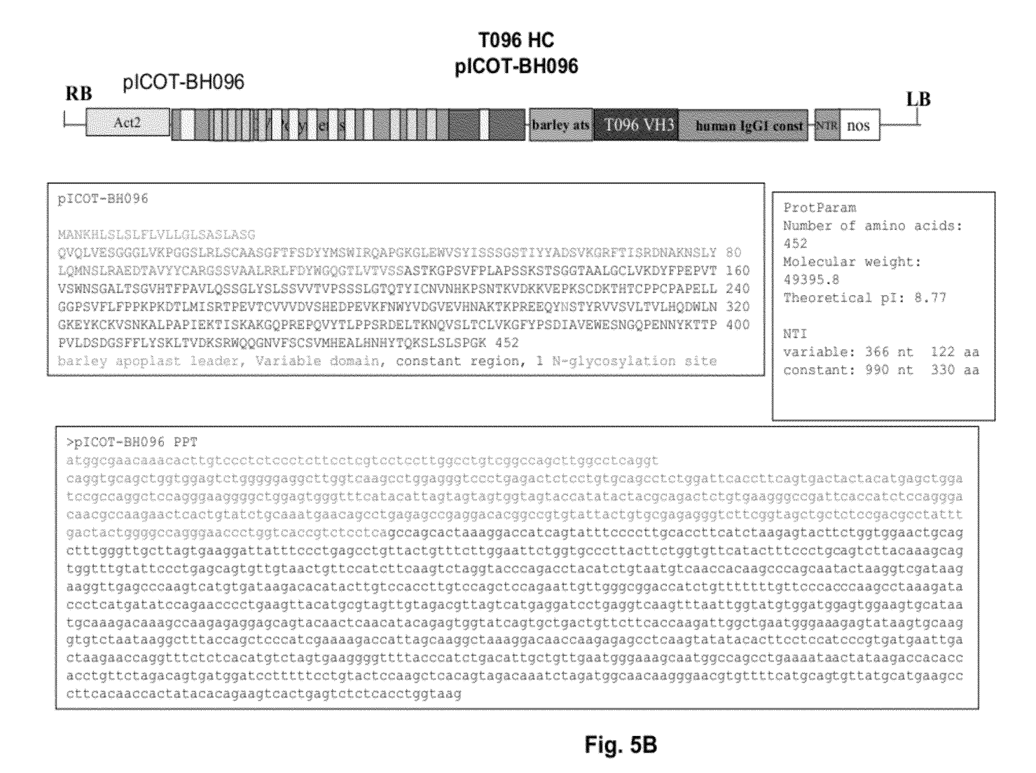

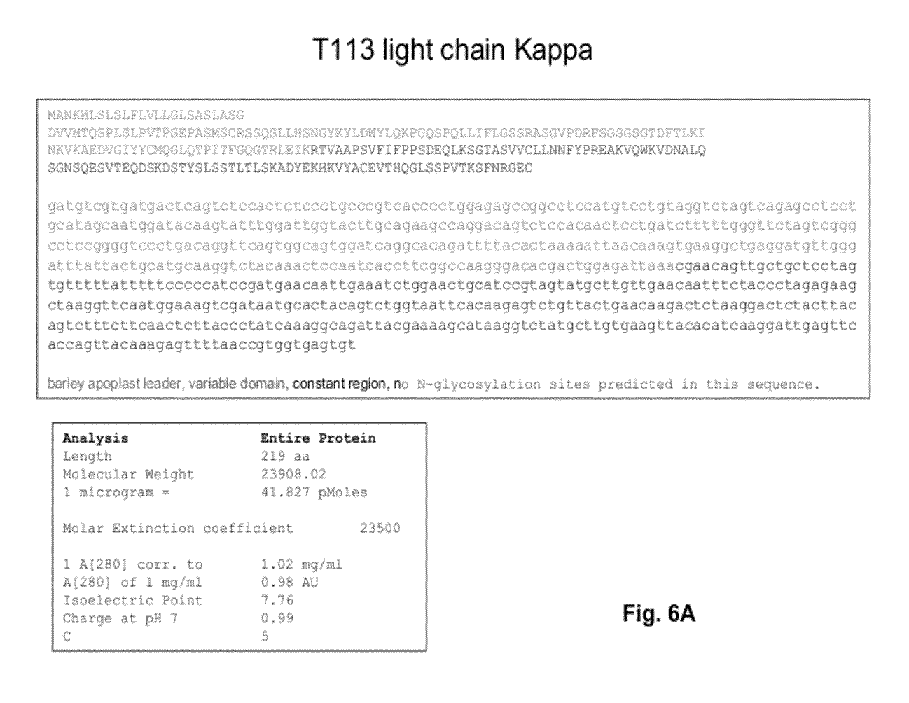

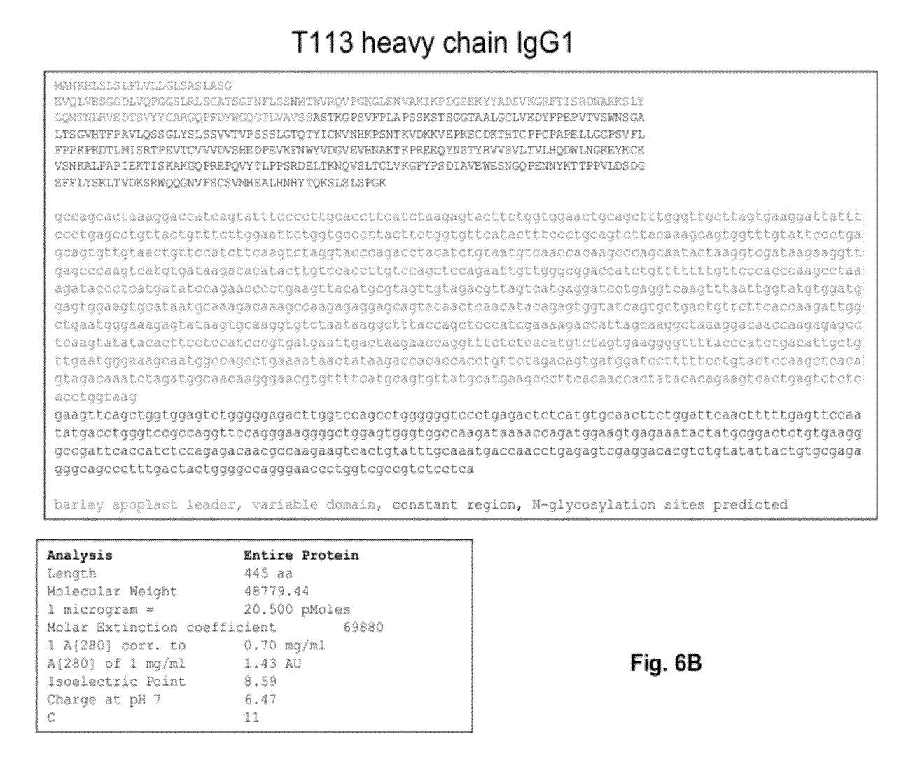

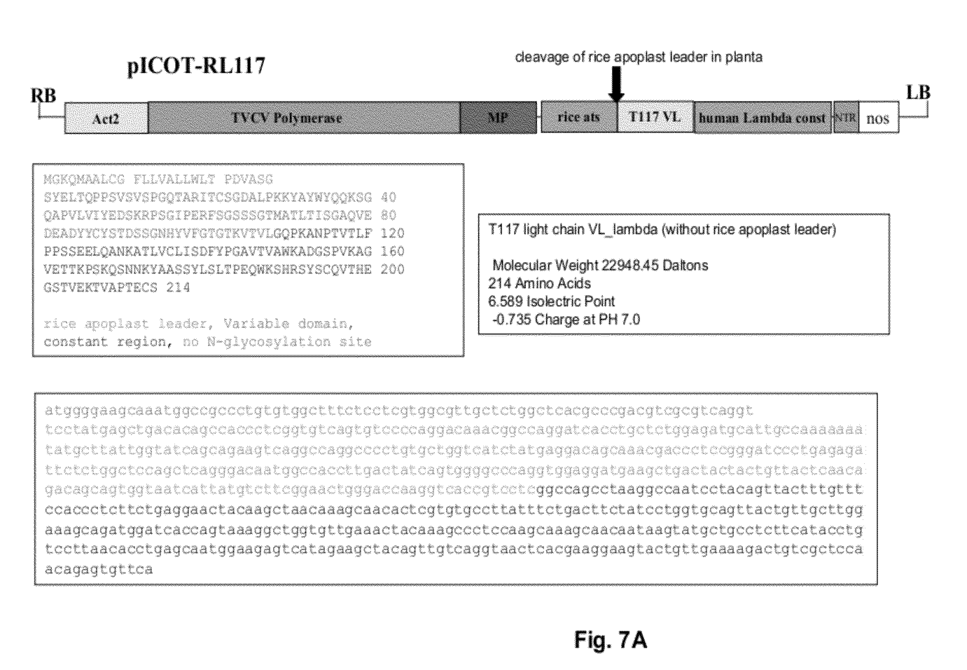

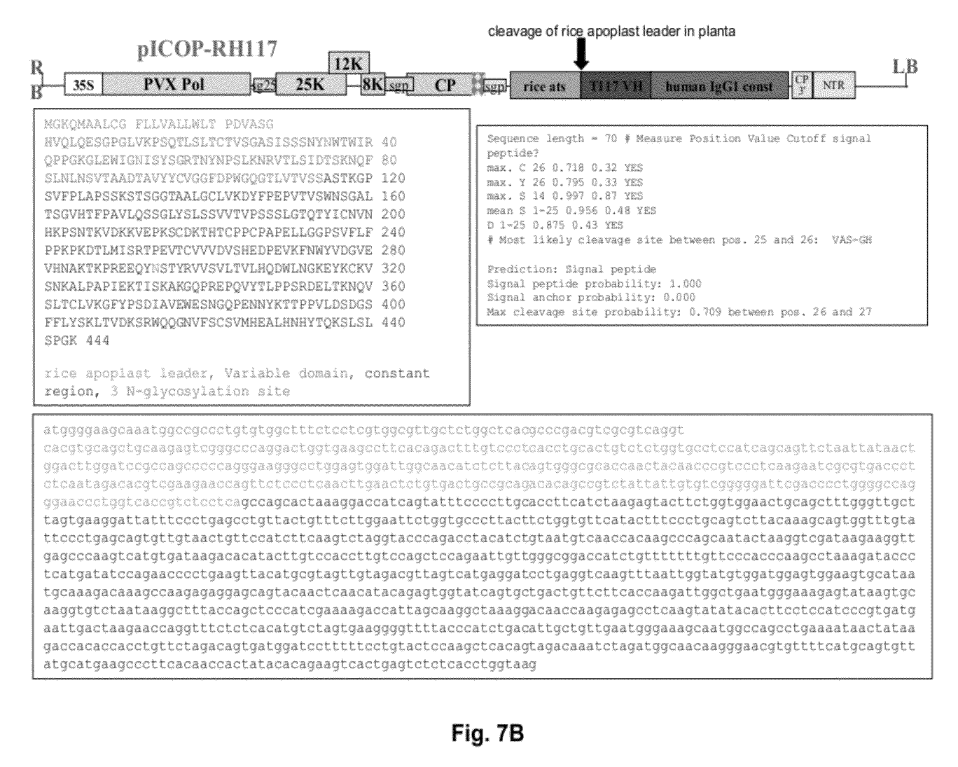

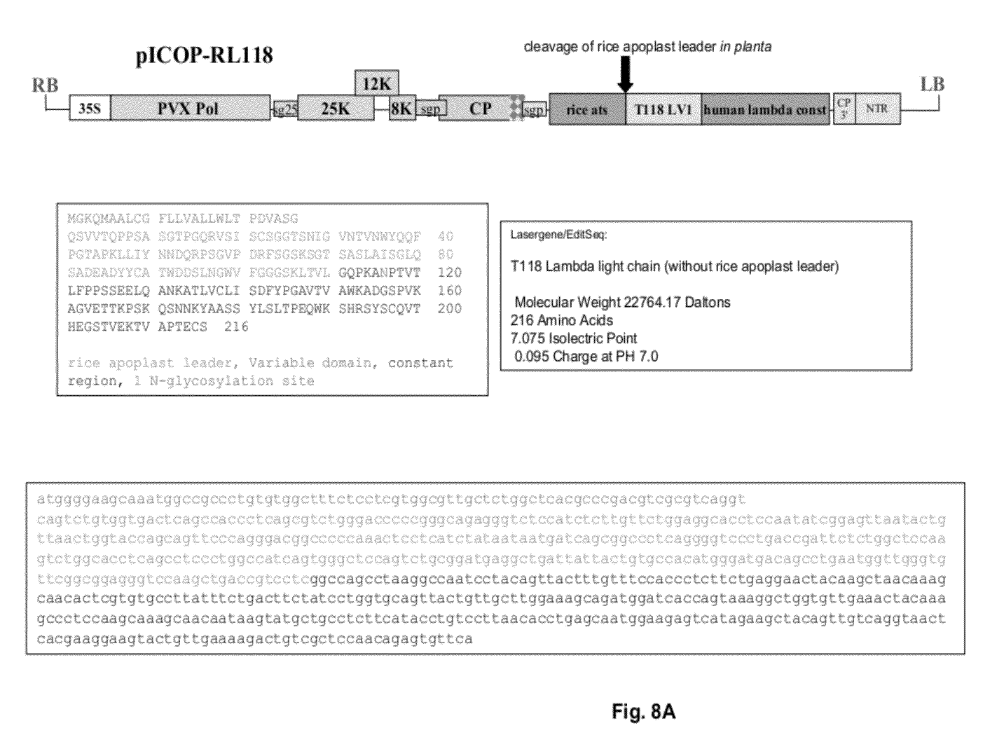

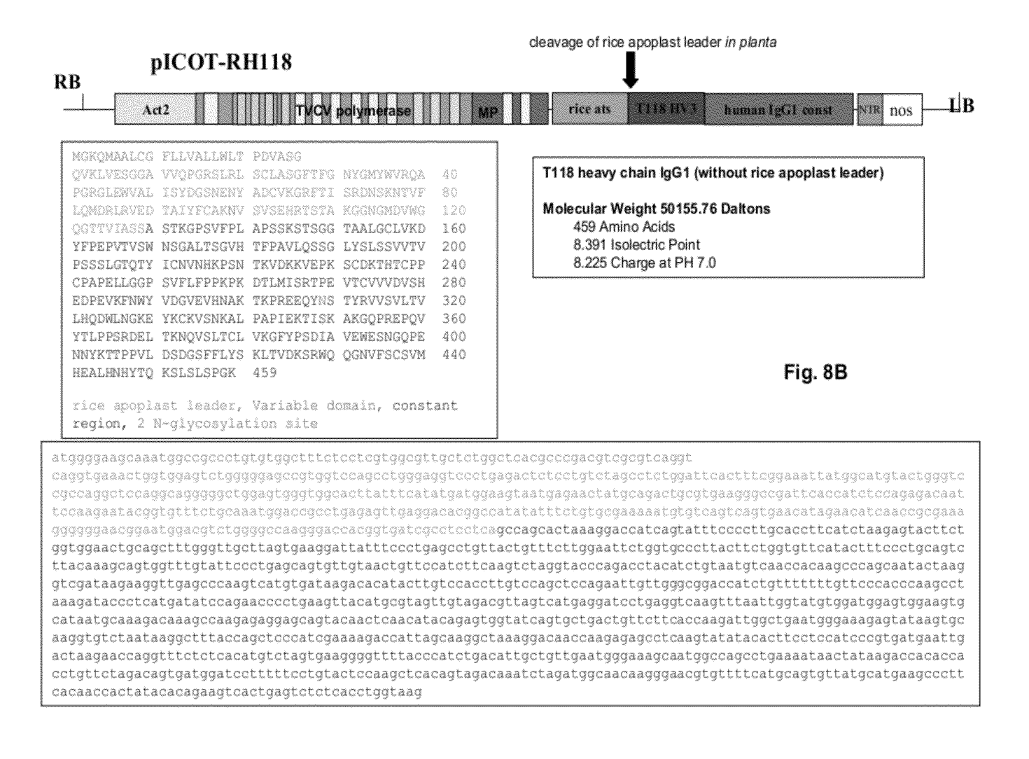

In the following, preparation of idiotypic antibodies for vaccination against NHL and expression in plants will be described. Cloning methods that can be used for cloning and expression of variable regions of Igs are described in WO 2010/040531 that is incorporated herein by reference. Notably, cloning of variable Ig regions is described in detail in Example 2 and with reference to FIG. 2 in WO 2010/040531. Simultaneous expression of heavy and light chains in plants can be done as described in WO2006/079546 or Giritch et al., Proc. Natl. Acad. Sci. USA 2006, 103 (40) 14701-14706.

### Expression Vector Preparation

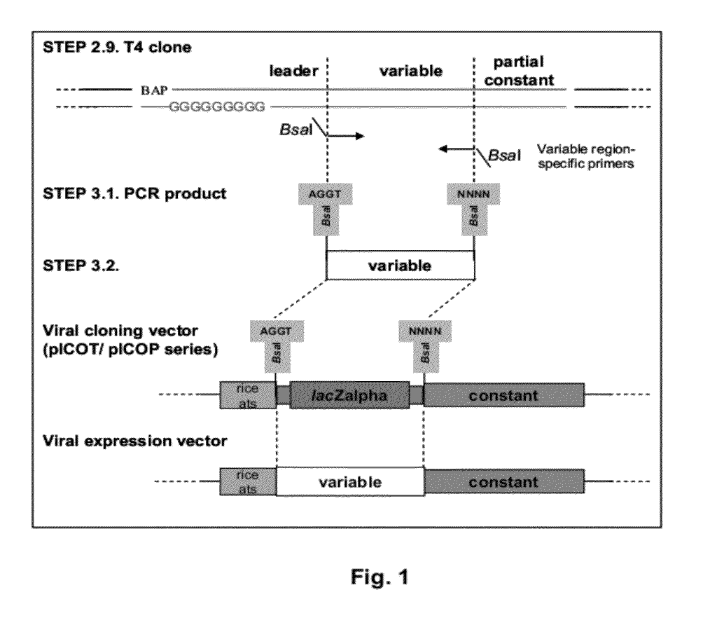

When the tumor sequence of the idiotypic antibody has been determined unequivocally (e.g. after cloning as described in Example 2 of WO 2010/040531), specific primers are designed to amplify the variable regions of the heavy and light chains for subsequent cloning in viral expression vectors of the pICOT (T for TMV) and pICOP (P for PVX) series. The variable region-specific primers (cf. FIG. 1) are designed to contain 6-10 codons of the variable region and the recognition site for the restriction enzyme BsaI, which allows direct cloning of the variable region in the viral expression vectors.

Two T4 clones (FIG. 1, top) are selected as PCR templates whose inserts represent the identified tumor idiotype, one for the light chain and one for the heavy chain. These clones originate from the tumor sequence identification steps of the process (cf. Example 2 of WO 2010/040531).

All amplified variable regions can be cloned in TMV and PVX expression vectors with a rice alpha-amylase signal peptide referred to as “ats” in the figures (an alternative thereto is barley amylase signal peptide). The cloning vectors for the heavy and light chains are TMV (qualified vector designation: pICOT series) and PVX (qualified vector designation: pICOP series) based vectors. Both contain two BsaI restriction sites located between a plant signal peptide and a plant codon-optimized immunoglobulin constant domain. The variable region of heavy and light chain can both be cloned in TMV and PVX vector, as both combinations (heavy chain in TMV and light chain in PVX, and heavy chain in PVX and light chain in TMV) will be tested for expression. The optimal combination of vectors is chosen for production.

### Cloning in Viral Expression Vectors

PCR products which should correspond to 300-400 bp in size are analyzed by agarose gel electrophoresis and column-purified prior to cloning in viral vectors. The PCR fragment is flanked by recognition sites for the class IIs restriction enzyme BsaI. This restriction enzyme is used because the sequence of the 4-nucleotide 5′ overhang obtained after digestion can be chosen to be any sequence of choice (the enzyme cuts outside of its recognition site). Therefore, the same enzyme can be used to digest both ends of the PCR product while producing overhangs that are different and therefore incompatible, but compatible with the target vector. Moreover, the recognition sites are eliminated after cloning, resulting in seamless junctions. See Engler et al. PLoS ONE, 2008; 3(11): e3647 for details on cloning methods using type IIs restriction enzymes.

The cloning vectors for the heavy and light chains are TMV (pICOT series) and PVX (pICOP series) based vectors. TMV stands for tobacco mosaic virus. PVX stands for potato virus X. Both types of cloning vectors contain two BsaI restriction sites located between a rice alpha-amylase signal peptide and a plant codon-optimized immunoglobulin constant domain, which can be, for example, IgG1, IgK, or IgL, resulting in a total of six different vectors. The cloning vectors also contain a lacZ-alpha gene for blue-white selection to facilitate screening of recombinant clones and a kanamycin-resistance cassette. The variable region of the heavy chain will be cloned in pICOT-RH and pICOP-RH, and the variable region of the light chain in either pICOT-RK and pICOP-RK if it belongs to a kappa chain, or in pICOT-RL and pICOP-RL if it belongs to a lambda chain. For an overview see FIGS. 1 and 2.

For cloning, vector and PCR product are digested with BsaI and ligated in a single reaction in a DNA thermocycler. The cloning reaction is transformed in chemo-competent E. coli DH10B and plated on LB agar medium containing X-gal and kanamycin and incubated overnight at 37° C. At least one clone per construct with correct sequence (alpha-amylase apoplast targeting signal, variable and constant region) is prepared for long-term storage as glycerol stock and the corresponding plasmid DNA can be transformed in Agrobacterium tumefaciens.

Transformation of Viral Expression Vectors into Agrobacterium tumefaciens

Plasmid DNA of all four viral expression vectors verified by sequencing is electroporated into Agrobacterium strain ICF 320, and the transformation plated on selective medium containing kanamycin (viral vector resistance marker), rifampicin (ICF 320 resistance marker) and X-gal (blue/white selection based on IacZ on the Ti plasmid of ICF 320). For an overview see FIG. 2. Plates are incubated for three to four days at 28° C. Transformants are analyzed by colony PCR using vector primers (pICOT-F and pICOT-R; pICOP-F and pICOP-R) designed to amplify the complete immunoglobulin chain. The PCR products are analyzed by agarose gel electrophoresis for correct size (light chain: 1.1 kb; heavy chain: 1.7 kb), column-purified and sequenced (alpha-amylase apoplast targeting signal, variable and constant region). Verified clones are then grown overnight at 28° C. in liquid LBS medium containing rifampicin and kanamycin for preparation of glycerol stock cultures and for expression testing.

Antibody Expression Test by Agro-Infiltration of Single N. benthamiana Plants and SDS-PAGE Analysis

To determine which vector combination (heavy chain in TMV or PVX and light chain in the complementary vector) provides the highest expression level, both combinations are infiltrated manually using a syringe without a needle on individual Nicotiana benthamiana plants (see FIG. 2). Plants are derived from the same seed bank as the ones used for production.

Overnight cultures of all constructs are diluted in infiltration buffer to a final OD₆₀₀=0.002 and the appropriate strains are mixed in one tube. The mixture is infiltrated in the lower side of upper leaves of different N. benthamiana plants. Plants are incubated in the greenhouse for about seven days, and infiltrated leaf areas harvested from different plants for each combination (approximately 100 mg fresh weight each). Proteins are extracted and the crude extract is analyzed using SDS-PAGE and Coomassie staining. The combination of vectors which results in the highest expression level is chosen, and the selected Agrobacterium strains used for upstream processing.

Igs expressed in plants can be isolated and purified according to generally known methods. For example, standard Protein A-based purification methods using commercial antibody purification kits can be used. Another method for Ig purification is described in WO2007/031339 using plant viral particles having bound affinity proteins such as protein A or derivatives displayed on the surface of the plant viral particles.

### EXAMPLES

### Example 1

### Preparation of Conjugates of Keyhole Limpet Hemocyanine and IgG Antibodies

0.5 mg/ml keyhole limpet hemocyanine (VACMUNE® liquid IEX from biosyn) and 0.5 mg/ml IgG antibody in PBS buffer pH 7.4 are mixed and cross-linked by adding 0.1% (v/v) glutardialdehyde. The mixture is incubated at 25° C. for 120 minutes. Then, the cross-linking reaction is quenched by adding 1% (v/v) 1 M aqueous glycine solution and incubating for 30 minutes. The protein conjugate solution is then frozen in liquid nitrogen until further use.

### Example 2

### Stability Tests Using Various KLH-IgG Conjugates

Conjugates T038 and T096 were rebuffered into the buffers shown below. The final conjugate concentration was 1 mg/ml. The obtained solutions were analysed by the Thermofluor method and by Micro DSC (differential scanning calorimetry) as well as by shaking and by applying shear forces. The following buffers were tested:
i. 20 mM citrate pH 5.0
ii. 20 mM citrate pH 6.0
iii. 20 mM citrate pH 6.6
iv. 20 mM histidine 7.0
v. 20 mM histidine pH 7.4
vi. 20 mM histidine pH 8.0

The conjugates were provided in PBS buffer after conjugation of the IgG antibodies to KLH (see Example 1. Size-exclusion chromatography (SEC) was performed on an ÄKTA™ Explorer (GE) system to yield the conjugate in the buffers listed above and indicated in the tables below.

The Thermofluor method was performed using a 750 Fast Real Time PCR System (Applied Biosystems). The formulations were added to a fluorescent dye in a 96 well-plate and were analyzed in the PCR System. The temperature was increased from 20° C. to 90° C. The melting points of proteins were determined by fluorescence detection.

Micro DSC was measured using a VP-DSC (GE Healthcare). The temperature was moved from 20° C. to 105° C. and the melting point of the protein was determined with the calorimeter.

The shear stress was generated by shaking on shaker (IKA, HS 260 control) into a climate chamber (MMM, FrioCell 200). The Critical Quality Parameter (aggregation) was decided after 1 h at 300 rpm and 20° C.

The stability of the solutions after the stress test was checked by noting the visual appearance. Further, dynamic light scattering (DLS) was performed using a Horiba LB550 (Retsch® Technology). The hydrodynamic diameter d_{H} (median) as determined by DLS are given in the tables.

The concentration c_{NHL} was measured by using a Nanodrop 2000 (Thermo Scientific). The NHL vaccines were calibrated at a wavelength of 280 nm.

The results are shown in the following Table 1. “Flocculation” means that individual flocculation particles could be seen by visual inspection.

TABLE 1

| | after ÄKTA | | | after stress test (1 h at 20° C., 300 rpm) | | |
|---|---|---|---|---|---|---|
| | visual | c_{NHL}* | d_{H} | visual | c_{NHL} | d_{H} |
| | appearance | [mg/ml] | [nm] | appearance | [mg/ml] | [nm] |
| NHL vaccine T096 | | | | | | |
| citrate pH 5.0 | slightly turbid | 0.11 | 3332 | — | — | — |
| citrate pH 6.0 | slightly turbid | 0.12 | 2427 | — | — | — |
| citrate pH 6.6 | slightly turbid | 0.15 | 199 | flocculation | 0.08 | 193 |
| histidine pH 7.0 | slightly turbid | 0.15 | 227 | flocculation | 0.17 | 190 |
| PBS pH 7.4 | slightly turbid | 0.18 | 199 | flocculation | 0.09 | 198 |
| histidine pH 8.0 | slightly turbid | 0.19 | 181 | no flocculation | 0.17 | 197 |
| NHL vaccine T038 | | | | | | |
| citrate pH 5.0 | clear | 0.22 | 2865 | — | — | — |
| citrate pH 6.0 | clear | 0.18 | 14 | — | — | — |
| citrate pH 6.6 | clear | 0.18 | 11 | flocculation | 0.13 | 12 |
| histidine pH 7.0 | clear | 0.19 | 16 | flocculation | 0.16 | 16 |
| PBS pH 7.4 | clear | 0.18 | 26 | flocculation | 0.14 | 9 |
| histidine pH 8.0 | clear | 0.20 | 15 | no flocculation | 0.19 | 11 |

The data in Table 1 show that both conjugates tested are not stable after application of shear stress in any buffer tested except histidine. In histidine, a pH above pH 7.0 achieved stability even upon application of shear stress.

### Example 3

### Stability Tests Using Various KLH-IgG Conjugates

The buffer of conjugates T069 and T096 was changed to 20 mM histidine pH 7.4, 0.01% Tween 80 during the workup of the conjugation reaction for storage. For the tests of this example, the buffers of the conjugates were changed to the following ones.
20 mM phosphate (Soerensen) pH 7.4
20 mM HEPES pH 7.4
20 mM TRIS pH 7.4
20 mM MOPS pH 7.4
20 mM histidine pH 7.4

These formulations were prepared with or without Tween 80 (0.1%), Pluronic® F68 (0.1%) or Tween 20 (0.1%). Filtration was performed using a membrane filter (Millipoore, Millex GV, filter unit 0.22 μm. Shear stress was applied by as described in Example 2. DSC assays were performed as described in Example 2 using a VP-Capillary DSC System (Microcal). Thermal stress was applied in differential scanning fluorimetry (DSF) experiments using ThermoFluor™ assays, where the impact of external factors on the stability of a protein in solution can be determined by measuring its unfolding temperature. This technology is based on the interaction of a hydrophobic fluorescent dye (Sypro Orange) with hydrophobic domains upon heating. C_{surf} indicates the surfactant concentration.

TABLE 2

| | | before | | after | | | |
|---|---|---|---|---|---|---|---|
| | | filtration | | filtration | | | |
| NHL vaccine T069 | C_{surf.} | visual | c_{NHL}* | visual | c_{NHL}* | d_{H} | |
| buffer | [%] | appear. | [mg/ml] | appear. | [mg/ml] | [nm] | Span |
| histidine pH 7.4 | 0.00 | clear | 0.97 | clear | 1.10 | 29 | 0.58 |
| HEPES pH 7.4 | 0.00 | clear | 0.94 | clear | 0.94 | 20 | 0.74 |
| TRIS pH 7.4 | 0.00 | clear | 0.90 | clear | 0.90 | 30 | 0.61 |
| phosphate pH 7.4 | 0.00 | clear | 1.00 | clear | 1.04 | 23 | 0.65 |
| MOPS pH 7.4 | 0.00 | clear | 0.83 | clear | 0.84 | 34 | 0.49 |
| | Tween 80 | | | | | | |
| histidine pH 7.4 | 0.01 | clear | — | clear | 1.08 | 31 | 0.5 |
| | Tween 80 | | | | | | |
| histidine pH 7.4 | 0.1 | clear | — | clear | 0.98 | — | — |
| HEPES pH 7.4 | 0.1 | clear | — | clear | 0.89 | 14 | 0.64 |
| TRIS pH 7.4 | 0.1 | clear | — | clear | 0.85 | 25 | 0.65 |
| phosphate pH 7.4 | 0.1 | clear | — | clear | 0.98 | 25 | 0.59 |
| MOPS pH 7.4 | 0.1 | clear | — | clear | 0.75 | — | — |
| | Tween 20 | | | | | | |
| histidine pH 7.4 | 0.1 | clear | — | clear | 0.97 | 19 | 0.74 |
| HEPES pH 7.4 | 0.1 | clear | — | clear | 0.86 | 24 | 0.62 |
| TRIS pH 7.4 | 0.1 | clear | — | clear | 0.82 | 29 | 0.51 |
| phosphate pH 7.4 | 0.1 | clear | — | clear | 0.95 | 23 | 0.60 |
| MOPS pH 7.4 | 0.1 | clear | — | clear | 0.75 | 29 | 0.49 |
| | F68 | | | | | | |
| histidine pH 7.4 | 0.1 | clear | — | clear | 0.98 | 29 | 0.64 |
| HEPES pH 7.4 | 0.1 | clear | — | clear | 0.86 | 27 | 0.64 |
| TRIS pH 7.4 | 0.1 | clear | — | clear | 0.82 | 23 | 0.63 |
| phosphate pH 7.4 | 0.1 | clear | — | clear | 0.93 | 25 | 0.59 |
| MOPS pH 7.4 | 0.1 | clear | — | clear | 0.75 | 25 | 0.68 |

TABLE 3

| | | thermal stress | | | | | | |
|---|---|---|---|---|---|---|---|---|
| | | DSF | DSC | | shear stress (32 h; 30° C., 300 rpm) | | | |
| NHL vaccine T069 | C_{surf.} | Tₘ₁ | Tₘ₁/Tₘ₂ | | visual | c_{NHL} | d_{H} | |
| buffer | [%] | [° C.] | [° C.] | | appear. | [%] | [nm] | Span |
| histidine pH 7.4 | 0.00 | 69.6 | 69.8 | 79.5 | clear | 144.2 | 36 | 0.57 |

| | | | | | | | |
|---|---|---|---|---|---|---|---|
| HEPES pH 7.4 | 0.00 | 68.8 | 69.0 | clear | 104.0 | 30 | 0.58 |
| TRIS pH 7.4 | 0.00 | 69.6 | — | clear | 103.3 | 37 | 0.54 |
| phosphate pH 7.4 | 0.00 | 68.1 | 69.3 | clear | 101.2 | 32 | 0.61 |
| MOPS pH 7.4 | 0.00 | 68.8 | 67.3 | clear | 103.4 | 36 | 0.54 |
| | Tween 80 | | | | | | |
| histidine pH 7.4 | 0.01 | —* | 79 | clear | 108.9 | 33 | 0.44 |
| | Tween 80 | | | | | | |
| histidine pH 7.4 | 0.1 | —* | — | clear | 108.3 | 34 | 0.46 |
| HEPES pH 7.4 | 0.1 | —* | 79.1 | clear | 115.0 | 26 | 0.66 |
| TRIS pH 7.4 | 0.1 | —* | — | clear | 104.1 | 28 | 0.64 |
| phosphate pH 7.4 | 0.1 | —* | 80.7 | clear | 100.4 | 31 | 0.56 |
| MOPS pH 7.4 | 0.1 | —* | — | clear | 108.7 | 25 | 0.70 |
| | Tween 20 | | | | | | |
| histidine pH 7.4 | 0.1 | —* | 79 | clear | 111.6 | 23 | 0.68 |
| HEPES pH 7.4 | 0.1 | —* | 67.6 | clear | 102.2 | 34 | 0.54 |
| TRIS pH 7.4 | 0.1 | —* | — | clear | 103.1 | 36 | 0.61 |
| phosphate pH 7.4 | 0.1 | —* | 66.5 | clear | 100.4 | 38 | 0.51 |
| MOPS pH 7.4 | 0.1 | —* | 68 | clear | 104.3 | 31 | 0.63 |
| | F68 | | | | | | |
| histidine pH 7.4 | 0.1 | 67.2 | 80.1 | clear | 112.6 | 23 | 0.71 |
| HEPES pH 7.4 | 0.1 | 66.1 | 73.7 | clear | 106.4 | 31 | 0.59 |
| TRIS pH 7.4 | 0.1 | 66.9 | 68.2 | clear | 103.2 | 36 | 0.60 |
| phosphate pH 7.4 | 0.1 | 62.9 | 67.6 | clear | 101.7 | 39 | 0.48 |
| MOPS pH 7.4 | 0.1 | 65.6 | 68.3 | clear | 103.8 | 36 | 0.57 |

*not measured since surfactants disturb the DSF measurement.

TABLE 4

| | | before | | after | | | |
|---|---|---|---|---|---|---|---|
| | | filtration | | filtration | | | |
| NHL vaccine T096 | C_{surf.} | visual | c_{NHL}* | visual | c_{NHL}* | d_{H} | |
| buffer | [%] | appear. | [mg/ml] | appear. | [mg/ml] | [nm] | Span |
| histidine pH 7.4 | 0.00 | opalescent | 0.97 | opalescent | 0.90 | 75 | 0.50 |
| HEPES pH 7.4 | 0.00 | opalescent | 0.98 | opalescent | 0.83 | 88 | 0.66 |
| TRIS pH 7.4 | 0.00 | turbid | 1.03 | opalescent | 0.64 | 95 | 0.61 |
| phosphate pH 7.4 | 0.00 | opalescent | 1.02 | opalescent | 0.92 | 72 | 0.59 |
| MOPS pH 7.4 | 0.00 | opalescent | 1.07 | opalescent | 0.98 | 96 | 0.53 |
| | Tween 80 | | | | | | |
| histidine pH 7.4 | 0.01 | clear solution | — | clear solution | 1.10 | 75 | 0.51 |
| | Tween 80 | | | | | | |
| histidine pH 7.4 | 0.1 | opalescent | — | opalescent | 0.95 | 75 | 0.52 |
| HEPES pH 7.4 | 0.1 | opalescent | — | opalescent | 0.88 | 87 | 0.79 |
| TRIS pH 7.4 | 0.1 | turbid | — | opalescent | 0.66 | 81 | 0.69 |
| phosphate pH 7.4 | 0.1 | opalescent | — | opalescent | 0.98 | 75 | 0.52 |
| MOPS pH 7.4 | 0.1 | opalescent | — | opalescent | 0.94 | 82 | 0.70 |
| | Tween 20 | | | | | | |
| histidine pH 7.4 | 0.1 | opalescent | — | opalescent | 0.92 | 73 | 0.54 |
| HEPES pH 7.4 | 0.1 | opalescent | — | opalescent | 0.84 | 96 | 0.58 |
| TRIS pH 7.4 | 0.1 | turbid | — | opalescent | 0.64 | 104 | 0.56 |
| phosphate pH 7.4 | 0.1 | opalescent | — | opalescent | 0.95 | — | — |
| MOPS pH 7.4 | 0.1 | opalescent | — | opalescent | 0.97 | 93 | 0.64 |
| | F68 | | | | | | |
| histidine pH 7.4 | 0.1 | opalescent | — | opalescent | 0.90 | 70 | 0.59 |
| HEPES pH 7.4 | 0.1 | opalescent | — | opalescent | 0.83 | 99 | 0.57 |
| TRIS pH 7.4 | 0.1 | turbid | — | opalescent | 0.64 | 92 | 0.64 |
| phosphate pH 7.4 | 0.1 | opalescent | — | opalescent | 0.92 | 74 | 0.59 |
| MOPS pH 7.4 | 0.1 | opalescent | — | opalescent | 1.07 | 97 | 0.57 |

*The measured data are referenced to calibration with the NHL vaccines into the transport buffer (10 mM NaH₂PO₄, 0.8% NaCl pH 7.3).

TABLE 5

| | | thermal stress | | | | | |
|---|---|---|---|---|---|---|---|
| | | DSF | DSC | shear stress (32 h; 30° C., 300 rpm) | | | |
| NHL vaccine T096 | C_{surf.} | Tₘ₁ | Tₘ₁ | visual | c_{NHL} | d_{H} | |
| buffer | [%] | [° C.] | [° C.] | appear. | [%] | [nm] | Span |
| histidine pH 7.4 | 0.00 | 75.2 | 77.9 | opalescent | 118.5 | 65 | 0.56 |
| HEPES pH 7.4 | 0.00 | 75.2 | 75.2 | opalescent | 102.7 | 136 | 0.77 |
| TRIS pH 7.4 | 0.00 | 75.4 | 74.9 | opalescent | 110.7 | 136 | 0.58 |
| phosphate pH 7.4 | 0.00 | 74.5 | 76.4 | opalescent | 101.1 | 80 | 0.51 |
| MOPS pH 7.4 | 0.00 | 75.4 | 74.7 | opalescent | 101.0 | 94 | 0.67 |
| | Tween 80 | | | | | | |
| histidine pH 7.4 | 0.01 | —* | — | opalescent | 107.9 | 71 | 0.55 |
| | Tween 80 | | | opalescent | | | |
| histidine pH 7.4 | 0.1 | —* | — | clear solution | 109.1 | 67 | 0.55 |
| HEPES pH 7.4 | 0.1 | —* | 79.9 | opalescent | 101.6 | 100 | 0.67 |
| TRIS pH 7.4 | 0.1 | —* | 70.0 | opalescent | 119.1 | 129 | 0.80 |
| phosphate pH 7.4 | 0.1 | —* | 83.4 | opalescent | 101.3 | 77 | 0.53 |
| MOPS pH 7.4 | 0.1 | —* | 84.9 | opalescent | 101.1 | 95 | 0.58 |
| | Tween 20 | | | | | | |
| histidine pH 7.4 | 0.1 | —* | 77.1 | opalescent | 106.2 | 66 | 0.57 |
| HEPES pH 7.4 | 0.1 | —* | 75.1 | opalescent | 100.8 | 99 | 0.67 |
| TRIS pH 7.4 | 0.1 | —* | 68.0 | opalescent | 111.0 | 145 | 0.57 |
| phosphate pH 7.4 | 0.1 | —* | 76.8 | opalescent | 101.3 | 82 | 0.47 |
| MOPS pH 7.4 | 0.1 | —* | 74.2 | opalescent | 102.2 | 95 | 0.61 |
| | F68 | | | | | | |
| Histidine pH 7.4 | 0.1 | 68.8 | 77.0 | opalescent | 109.1 | 73 | 0.52 |
| HEPES pH 7.4 | 0.1 | 59.1/70.4 | 75.2 | opalescent | 105.1 | 98 | 0.60 |
| TRIS pH 7.4 | 0.1 | 58.1/71.2 | 74.9 | opalescent | 119.6 | 186 | 0.77 |
| phosphate pH 7.4 | 0.1 | 66.9 | 75 | opalescent | 101.5 | 86 | 0.49 |
| MOPS pH 7.4 | 0.1 | 75.1 | 75.4 | opalescent | 104.3 | 99 | 0.55 |

*surfactants disturb the DSF measurement

### Example 4

### Tests Using Further Conjugates

In the above examples, histidine buffer showed the best stabilisation at pH 7.4. Histidine buffer was further tested in the presence of 0.01% Tween 80 with six further conjugates. The conjugates used were T038, T069, T096, T113, T117, T118 at the concentration of 1 mg/ml. Solutions were isotonised with trehalose. The test solutions were made by exchanging a phosphate buffer used for purification of the conjugates into 20 mM histidine pH 7.4 mit 0.01% Tween 80, trehalose. Tests were performed as described above.

TABLE 6

| | after | | | | | |
|---|---|---|---|---|---|---|
| | ÄKTA | | | stress test (≧48 h 20° C., 300 rpm) | | |
| NHL | visual | c_{NHL}* | d_{H} | visual | c_{NHL} | d_{H} |
| vaccine | appear. | [mg/ml] | [nm] | appear. | [mg/ml] | [nm] |
| T038 | clear | 1.45 | 40 | unchanged | 1.46 | 46 |
| T069 | clear | 0.94 | 95 | unchanged | 1.03 | 93 |
| T096 | slightly | 1.32 | 110 | unchanged | 1.31 | 93 |
| | turbid | | | | | |
| T113 | clear | 0.97 | 114 | unchanged | 0.94 | 114 |
| T117 | clear | 0.66 | 76 | unchanged | 0.72 | 70 |
| T118 | clear | 1.03 | 32 | unchanged | 1.02 | 31 |

*The measured data are referenced to calibration with the NHL vaccines into the transport buffer (10 mM NaH₂PO₄, 0.8% NaCl pH 7.3).

The data of Table 6 show that a buffer containing 20 mM histidine, 0.01% Tween 80 stabilises all conjugates tested against temperature and shear stress.

### Example 5

### Stability Tests Using Surfactants

Conjugate T038 was tested in 20 mM histidine pH 7.4 containing various surfactants. The following surfactants were separately tested in this buffer: Tween80 (Polysorbat 80), Tween20 (Polysorbat 20), Synperonic F68. Additionally, HP-β-CD was used as alternative stabiliser. Results are shown in the following tables.

TABLE 7

| NHL Va. T038 | | | | | |
|---|---|---|---|---|---|
| 20 mM His | | after ÄKTA | | | |
| pH 7.4 | C_{surf.} | visual | c_{NHL}* | d_{H} | |
| surfactant | [%] | appear. | [mg/ml] | [nm] | Span |
| Tween80 | 0.01 | clear sol. | 1.45 | 40 | 0.63 |
| Tween20 | 0.01 | clear sol. | 0.97 | 56 | 0.51 |
| F68 | 0.01 | clear sol. | 1.11 | 55 | 0.50 |
| HP-β-CD | 0.40 | clear sol. | 1.02 | — | — |
| NHL Va. T038 | | shear stress | | | |
| 20 mM His | | (48 h, 20° C.; 300 rpm) | | | |
| pH 7.4 | C_{surf.} | visual | c_{NHL}* | d_{H} | |
| surfactant | [%] | appear. | [%] | [nm] | Span |
| Tween80 | 0.01 | clear sol. | 98.1 | 51 | 0.45 |
| Tween20 | 0.01 | clear sol. | 106.3 | 55 | 0.52 |
| F68 | 0.01 | clear sol. | 90.1 | 45 | 0.55 |
| HP-β-CD | 0.40 | clear sol. | 92.7 | 46 | 0.53 |

### Annex

### Keyhole Limpet Hemocyanin 1 (KLH1)

GenBank entry Q53IP9
3408 aa, calculated molecular weight: 391.5 kDa

```
SEQ ID NO: 1:
                10 20 30 40 50 60
MLSVRLLIVV LALANAENLV RKSVEHLTQE ETLDLQAALR ELQMDSSSIG FQKIAAAHGA
                70 80 90 100 110 120
PASCVHKDTS IACCIHGMPT FPHWHRAYVV HMERALQTKR RTSGLPYWDW TEPITQLPSL
              130 140 150 160 170 180
AADPVYIDSQ GGKAHTNYWY RGNIGFLDKK TNRAADDRLF EKVKPGQHTH LMESVLDPLE
              190 200 210 220 230 240
QDEFCKFEIQ FELPHNAIHY LVGGKHDYSM ANLEYTAYDP IFFLHHSNVD RIFAIWQRLQ
              250 260 270 280 290 300
ELRNKDPKAM DCAQELLHQK MEPFSWEDND IPLTNDYDTL NLNGMTPEEL KTYLDERSSR
              310 320 330 340 350 360
ARAFASFRLK GFGGSANVFV YVCIPDDNDR NDDHCEKAGD FFVLGGPSEM KWQFYRPYLF
              370 380 390 400 410 420
DLSDTVHKMG MKLDGHYTVK AELFSVNGTA LPDDLLPHPV VVHHPEKGFT DPPVKHHQSA
              430 440 450 460 470 480
NLLVRKNIND LTREEVLNLR EAFHKFQEDR SVDGYQATAE YHGLPARCPR PDAKDRYACC
              490 500 510 520 530 540
VHGMPIFPHW HRLFVTQVED ALVGRGATIG IPYWGLPYWD WTEPMTHIPG LAGNKTYVDS
              550 560 570 580 590 600
HGASHTNPFH SSVIAFEENA PHTKRQIDQR LFKPATFGHH TDLFNQILYA FEQEDYCDFE
              610 620 630 640 650 660
VQFEITHNTI HAWTGGSEHF SMSSLHYTAF DPLFYFHHSN VDRLWAVWQA LQMRRHKPYR
              670 680 690 700 710 720
AHCAISLEHM HLKPFAFSSP LNNNEKTHAN AMPNKIYDYE NVLHYTYEDL TFGGISLENI
              730 740 750 760 770 780
EKMIHENQQE DRIYAGFLLA GIRTSANVDI FIKTTDSVQH KAGTFAVLGG SKEMKWGFDR
              790 800 810 820 830 840
VFKFDITHVL KDLDLTADGD FEVTVDITEV DGTKLASSLI PHASVIREHA RGKLNRVKFD
              850 860 870 880 890 900
KVPRSRLIRK NVDRLSPEEM NELRKALALL KEDKSAGGFQ QLGAFHGEPK WCPSPEASKK
              910 920 930 940 950 960
FACCVHGMSV FPHWHRLLTV QSENALRRHG YDGALPYWDW TSPLNHLPEL ADHEKYVDPE
              970 980 990 1000 1010 1020
DGVEKHNPWF DGHIDTVDKT TTRSVQNKLF EQPEFGHYTS IAKQVLLALE QDNFCDFEIQ
            1030 1040 1050 1060 1070 1080
YEIAHNYIHA LVGGAQPYGM ASLRYTAFDP LFYLHHSNTD RIWAIWQALQ KYRGKPYNVA
            1090 1100 1110 1120 1130 1140
NCAVTSMREP LQPFGLSANI NTDHVTKEHS VPFNVFDYKT NFNYEYDTLE FNGLSISQLN
            1150 1160 1170 1180 1190 1200
KKLEAIKSQD RFFAGFLLSG FKKSSLVKFN ICTDSSNCHP AGEFYLLGDE NEMPWAYDRV
            1210 1220 1230 1240 1250 1260
FKYDITEKLH DLKLHAEDHF YIDYEVFDLK PASLGKDLFK QPSVIHEPRI GHHEGEVYQA
            1270 1280 1290 1300 1310 1320
EVTSANRIRK NIENLSLGEL ESLRAAFLEI ENDGTYESIA KFHGSPGLCQ LNGNPISCCV
            1330 1340 1350 1360 1370 1380
HGMPTFPHWH RLYVVVVENA LLKKGSSVAV PYWDWTKRIE HLPHLISDAT YYNSRQHHYE
            1390 1400 1410 1420 1430 1440
TNPFHHGKIT HENEITTRDP KDSLFHSDYF YEQVLYALEQ DNFCDFEIQL EILHNALHSL
            1450 1460 1470 1480 1490 1500
LGGKGKYSMS NLDYAAFDPV FFLHHATTDR IWAIWQDLQR FRKRPYREAN CAIQLMHTPL
            1510 1520 1530 1540 1550 1560
QPFDKSDNND EATKTHATPH DGFEYQNSFG YAYDNLELNH YSIPQLDHML QERKRHDRVF
            1570 1580 1590 1600 1610 1620
AGFLLHNIGT SADGHVFVCL PTGEHTKDCS HEAGMFSILG GQTEMSFVFD RLYKLDITKA
            1630 1640 1650 1660 1670 1680
LKKNGVHLQG DFDLEIEITA VNGSHLDSHV IHSPTILFEA GTDSAHTDDG HTEPVMIRKD
            1690 1700 1710 1720 1730 1740
ITQLDKRQQL SLVKALESMK ADHSSDGFQA IASFHALPPL CPSPAASKRF ACCVHGMATF
            1750 1760 1770 1780 1790 1800
PQWHRLYTVQ FQDSLRKHGA VVGLPYWDWT LPRSELPELL TVSTIHDPET GRDIPNPFIG
            1810 1820 1830 1840 1850 1860
SKIEFEGENV HTKRDINRDR LFQGSTKTHH NWFIEQALLA LEQTNYCDFE VQFEIMHNGV
            1870 1880 1890 1900 1910 1920
HTWVGGKEPY GIGHLHYASY DPLFYIHHSQ TDRIWAIWQS LQRFRGLSGS EANCAVNLMK
            1930 1940 1950 1960 1970 1980
TPLKPFSFGA PYNLNDHTHD FSKPEDTFDY QKFGYIYDTL EFAGWSIRGI DHIVRNRQEH
            1990 2000 2010 2020 2030 2040
SRVFAGFLLE GFGTSATVDF QVCRTAGDCE DAGYFTVLGG EKEMPWAFDR LYKYDITETL
            2050 2060 2070 2080 2090 2100
DKMNLRHDEI FQIEVTITSY DGTVLDSGLI PTPSIIYDPA HHDISSHHLS LNKVRHDLST
            2110 2120 2130 2140 2150 2160
LSERDIGSLK YALSSLQADT SADGFAAIAS FHGLPAKCND SHNNEVACCI HGMPTFPHWH
            2170 2180 2190 2200 2210 2220
RLYTLQFEQA LRRHGSSVAV PYWDWTKPIH NIPHLFTDKE YYDVWRNKVM PNPFARGYVP
            2230 2240 2250 2260 2270 2280
SHDTYTVRDV QEGLFHLTST GEHSALLNQA LLALEQHDYC DFAVQFEVMH NTIHYLVGGP
            2290 2300 2310 2320 2330 2340
QVYSLSSLHY ASYDPIFFIH HSFVDKVWAV WQALQEKRGL PSDRADCAVS LMTQNMRPFH
            2350 2360 2370 2380 2390 2400
YEINHNQFTK KHAVPNDVFK YELLGYRYDN LEIGGMNLHE IEKEIKDKQH HVRVFAGFLL
            2410 2420 2430 2440 2450 2460
HGIRTSADVQ FQICKTSEDC HHGGQIFVLG GTKEMAWAYN RLFKYDITHA LHDAHITPED
            2470 2480 2490 2500 2510 2520
VFHPSEPFFI KVSVTAVNGT VLPASILHAP TIIYEPGLDH HEDHHSSSMA GHGVRKEINT
            2530 2540 2550 2560 2570 2580
LTTAEVDNLK DAMRAVMADH GPNGYQAIAA FHGNPPMCPM PDGKNYSCCT HGMATFPHWH
            2590 2600 2610 2620 2630 2640
RLYTKQMEDA LTAHGARVGL PYWDGTTAFT ALPTFVTDEE DNPFHHGHID YLGVDTTRSP
            2650 2660 2670 2680 2690 2700
RDKLFNDPER GSESFFYRQV LLALEQTDFC QFEVQFEITH NAIHSWTGGL TPYGMSTLEY
            2710 2720 2730 2740 2750 2760
TTYDPLFWLH HANTDRIWAI WQALQEYRGL PYDHANCEIQ AMKRPLRPFS DPINHNAFTH
            2770 2780 2790 2800 2810 2820
SNAKPTDVFE YSRFNFQYDN LRFHGMTIKK LEHELEKQKE EDRTFAAFLL HGIKKSADVS
            2830 2840 2850 2860 2870 2880
FDVCNHDGEC HFAGTFAILG GEHEMPWSFD RLFRYDITQV LKQMHLEYDS DFTFHMRIID
            2890 2900 2910 2920 2930 2940
TSGKQLPSDL IKMPTVEHSP GGKHHEKHHE DHHEDILVRK NIHSLSHHEA EELRDALYKL
            2950 2960 2970 2980 2990 3000
QNDESHGGYE HIAGFHGYPN LCPEKGDEKY PCCVHGMSIF PHWHRLHTIQ FERALKKHGS
            3010 3020 3030 3040 3050 3060
HLGIPYWDWT QTISSLPTFF ADSGNNNPFF KYHIRSINQD TVRDVNEAIF QQTKFGEFSS
            3070 3080 3090 3100 3110 3120
IFYLALQALE EDNYCDFEVQ YEILHNEVHA LIGGAEKYSM STLEYSAFDP YFMIHHASLD
            3130 3140 3150 3160 3170 3180
KIWIIWQELQ KRRVKPAHAG SCAGDIMHVP LHPFNYESVN NDDFTRENSL PNAVVDSHRF
            3190 3200 3210 3220 3230 3240
NYKYDNLNLH GHNIEELEEV LRSLRLKSRV FAGFVLSGIR TTAVVKVYIK SGTDSDDEYA
            3250 3260 3270 3280 3290 3300
GSFVILGGAK EMPWAYERLY RFDITETVHN LNLTDDHVKF RFDLKKYDHT ELDASVLPAP
            3310 3320 3330 3340 3350 3360
IIVRRPNNAV FDIIEIPIGK DVNLPPKVVV KRGTKIMFMS VDEAVTTPML NLGSYTAMFK
            3370 3380 3390 3400
CKVPPFSFHA FELGKMYSVE SGDYFMTAST TELCNDNNLR IHVHVDDE
```

### Keyhole Limpet Hemocyanin 2 (KLH2)

Gen Bank entry CAG28308
3421 aa, calculated molecular weight: 391.5 kDa

```
SEQ ID NO: 2:
                10 20 30 40 50 60
MWTILALLTA TLLFEGAFSV DTVVRKNVDS LSSDEVLALE KALDDLQQDD SNQGYQAIAG
                70 80 90 100 110 120
YHGVPTMCVD KHEKNVACCL HGMPSFPLWH RLYVVQLERA LIRKKATISI PYWDWTSELT
              130 140 150 160 170 180
HLPELVSHPL FVGTEGGKAH DNSWYRADIT FLNKKTSRAV DDRLFEKVQP GHHTRLMEGI
              190 200 210 220 230 240
LDALEQDEFC KFEIQFELAH NAIHYLVGGR HTYSMSHLEY TSYDPLFFLH HSNTDRIFAI
              250 260 270 280 290 300
WQRLQQLRGK DPNSADCAHN LIHTPMEPFD RDTNPLDLTR EHAKPADSFD YGRLGYQYDD
              310 320 330 340 350 360
LSLNGMSPEE LNVYLGERAA KERTFASFIL SGFGGSANVV VYVCRPAHDE ISDDQCIKAG
              370 380 390 400 410 420
DFFLLGGPTE MKWGFYRAYH FDVTDSVASI DDDGHGHYYV KSELFSVNGS ALSNDILRQP
              430 440 450 460 470 480
TLVHRPAKGH FDKPPVPVAQ ANLAVRKNIN DLTAEETYSL RKAMERFQND KSVDGYQATV
              490 500 510 520 530 540
EFHALPARCP RPDAKDRFAC CVHGMATFPH WHRLFVTQVE DALLRRGSTI GLPNWDWTMP
              550 560 570 580 590 600
MDHLPELATS ETYLDPVTGE TKNNPFHHAQ VAFENGVTSR NPDAKLFMKP TYGDHTYLFD
              610 620 630 640 650 660
SMIYAFEQED FCDFEVQYEL THNAIHAWVG GSEKYSMSSL HYTAFDPIFY LHHSNVDRLW
              670 680 690 700 710 720
AIWQALQIRR GKSYKAHCAS SQEREPLKPF AFSSPLNNNE KTYHNSVPTN VYDYVGVLHY
              730 740 750 760 770 780
RYDDLQFGGM TMSELEEYIH KQTQHDRTFA GFFLSYIGTS ASVDIFINRE GHDKYKVGSF
              790 800 810 820 830 840
VVLGGSKEMK WGFDRMYKYE ITEALKTLNV AVDDGFSITV EITDVDGSPP SADLIPPPAI
              850 860 870 880 890 900
IFERADAKDF GHSRKIRKAV DSLTVEEQTS LRRAMADLQD DKTSGGFQQI AAFHGEPKWC
              910 920 930 940 950 960
PSPEAEKKFA CCVHGMAVFP HWHRLLTVQG ENALRKHGFT GGLPYWDWTR SMSALPHFVA
              970 980 990 1000 1010 1020
DPTYNDAISS QEEDNPWHHG HIDSVGHDTT RDVRDDLYQS PGFGHYTDIA KQVLLAFEQD
            1030 1040 1050 1060 1070 1080
DFCDFEVQFE IAHNFIHALV GGNEPYSMSS LRYTTYDPIF FLHRSNTDRL WAIWQALQKY
            1090 1100 1110 1120 1130 1140
RGKPYNTANC AIASMRKPLQ PFGLDSVINP DDETREHSVP FRVFDYKNNF DYEYESLAFN
            1150 1160 1170 1180 1190 1200
GLSIAQLDRE LQRRKSHDRV FAGFLLHEIG QSALVKFYVC KHNVSDCDHY AGEFYILGDE
            1210 1220 1230 1240 1250 1260
AEMPWRYDRV YKYEITQQLH DLDLHVGDNF FLKYEAFDLN GGSLGGSIFS QPSVIFEPAA
            1270 1280 1290 1300 1310 1320
GSHQADEYRE AVTSASHIRK NIRDLSEGEI ESIRSAFLQI QKEGIYENIA KFHGKPGLCE
            1330 1340 1350 1360 1370 1380
HDGHPVACCV HGMPTFPHWH RLYVLQVENA LLERGSAVAV PYWDWTEKAD SLPSLINDAT
            1390 1400 1410 1420 1430 1440
YFNSRSQTFD PNPFFRGHIA FENAVTSRDP QPELWDNKDF YENVMLALEQ DNFCDFEIQL
            1450 1460 1470 1480 1490 1500
ELIHNALHSR LGGRAKYSLS SLDYTAFDPV FFLHHANVDR IWAIWQDLQR YRKKPYNEAD
            1510 1520 1530 1540 1550 1560
CAVNEMRKPL QPFNNPELNS DSMTLKHNLP QDSFDYQNRF RYQYDNLQFN HFSIQKLDQT
            1570 1580 1590 1600 1610 1620
IQARKQHDRV FAGFILHNIG TSAVVDIYIC VEQGGEQNCK TKAGSFTILG GETEMPFHFD
            1630 1640 1650 1660 1670 1680
RLYKFDITSA LHKLGVPLDG HGFDIKVDVR AVNGSHLDQH ILNEPSLLFV PGERKNIYYD
            1690 1700 1710 1720 1730 1740
GLSQHNLVRK EVSSLTTLEK HFLRKALKNM QADDSPDGYQ AIASFHALPP LCPSPSAAHR
            1750 1760 1770 1780 1790 1800
HACCLHGMAT FPQWHRLYTV QFEDSLKRHG SIVGLPYWDW LKPQSALPDL VTQETYEHLF
            1810 1820 1830 1840 1850 1860
SHKTFPNPFL KANIEFEGEG VTTERDVDAE HLFAKGNLVY NNWFCNQALY ALEQENYCDF
            1870 1880 1890 1900 1910 1920
EIQFEILHNG IHSWVGGSKT HSIGHLHYAS YDPLFYIHHS QTDRIWAIWQ ALQEHRGLSG
            1930 1940 1950 1960 1970 1980
KEAHCALEQM KDPLKPFSFG SPYNLNKRTQ EFSKPEDTFD YHRFGYEYDS LEFVGMSVSS
            1990 2000 2010 2020 2030 2040
LHNYIKQQQE ADRVFAGFLL KGFGQSASVS FDICRPDQSC QEAGYFSVLG GSSEMPWQFD
            2050 2060 2070 2080 2090 2100
RLYKYDITKT LKDMKLRYDD TFTIKVHIKD IAGAELDSDL IPTPSVLLEE GKHGINVRHV
            2110 2120 2130 2140 2150 2160
GRNRIRMELS ELTERDLASL KSAMRSLQAD DGVNGYQAIA SFHGLPASCH DDEGHEIACC
            2170 2180 2190 2200 2210 2220
IHGMPVFPHW HRLYTLQMDM ALLSHGSAVA IPYWDWTKPI SKLPDLFTSP EYYDPWRDAV
            2230 2240 2250 2260 2270 2280
VNNPFAKGYI KSEDAYTVRD PQDILYHLQD ETGTSVLLDQ TLLALEQTDF CDFEVQFEVV
            2290 2300 2310 2320 2330 2340
HNAIHYLVGG RQVYALSSQH YASYDPAFFI HHSFVDKIWA VWQALQKKRK RPYHKADCAL
            2350 2360 2370 2380 2390 2400
NMMTKPMRPF AHDFNHNGFT KMHAVPNTLF DFQDLFYTYD NLEIAGMNVN QLEAEINRRK
            2410 2420 2430 2440 2450 2460
SQTRVFAGFL LHGIGRSADV RFWICKTADD CHASGMIFIL GGSKEMHWAY DRNFKYDITQ
            2470 2480 2490 2500 2510 2520
ALKAQSIHPE DVFDTDAPFF IKVEVHGVNK TALPSSAIPA PTIIYSAGEG HTDDHGSDHI
            2530 2540 2550 2560 2570 2580
AGSGVRKDVT SLTASEIENL RHALQSVMDD DGPNGFQAIA AYHGSPPMCH MHDGRDVACC
            2590 2600 2610 2620 2630 2640
THGMASFPHW HRLFVKQMED ALAAHGAHIG IPYWDWTSAF SHLPALVTDH EHNPFHHGHI
            2650 2660 2670 2680 2690 2700
AHRNVDTSRS PRDMLFNDPE HGSESFFYRQ VLLALEQTDF CQFEVQFEIT HNAIHSWTGG
            2710 2720 2730 2740 2750 2760
HTPYGMSSLE YTAYDPLFYL HHSNTDRIWA IWQALQKYRG FQYNAAHCDI QVLKQPLKPF
            2770 2780 2790 2800 2810 2820
SESRNPNPVT RANSRAVDSF DYERLNYQYD TLTFHGHSIS ELDAMLQERK KEERTFAAFL
            2830 2840 2850 2860 2870 2880
LHGFGASADV SFDVCTPDGH CAFAGTFAVL GGELEMPWSF ERLFRYDITK VLKQMNLHYD
            2890 2900 2910 2920 2930 2940
SEFHFELKIV GTDGTELPSD RIKSPTIEHH GGDHHGGDTS GHDHSERHDG FFRKEVGSLS
            2950 2960 2970 2980 2990 3000
LDEANDLKNA LYKLQNDQGP NGYESIAGYH GYPFLCPEHG EDQYACCVHG MPVFPHWHRL
            3010 3020 3030 3040 3050 3060
HTIQFERALK EHGSHLGIPY WDWTKSMIAL PAFFADSSNS NPFYKYHIMK AGHDTARSPS
            3070 3080 3090 3100 3110 3120
DLLFNQPQLH GYDYLYYLAL STLEEDNYCD FEVHYEILHN AVHLWLGGTE TYSMSSLAFS
            3130 3140 3150 3160 3170 3180
AYDPVFMILH SGLDRLWIIW QELQKLRKKP YNAAKCAGHM MDEPLHPFNY ESANHDSFTR
            3190 3200 3210 3220 3230 3240
ANAKPSTVFD SHKFNYHYDN PDVRGNSIQE ISAIIHDLRN QPRVFAGFVL SGIYTSANVK
            3250 3260 3270 3280 3290 3300
IYLVREGHDD ENVGSFVVLG GPKEMPWAYE RIFKYDITEV ANRLNMHHDD TFNFRLEVQS
            3310 3320 3330 3340 3350 3360
YTGEMVTHHL PEPLIIYRPA KQEYDVLVIP LGSGHKLPPK VIVKRGTRIM FHPVDDTVNR
            3370 3380 3390 3400 3410 3420
PVVDLGSHTA LYNCVVPPFT YNGYELDHAY SLRDGHYYIA GPTKDLCTSG NVRIHIHIED
E
```

“Keyhole limpet hemocyanin: Structural and functional characterization of two different subunits and multimers”; Swerdlow R D et al.; Comparative Biochemistry and Physiology Part B: Biochemistry and Molecular Biology, Volume 113, Issue 3, March 1996, Pages 537-548

1. A pharmaceutical preparation comprising a protein conjugate comprising an immunoglobulin conjugated to a carrier protein, and histidine as a buffering agent.

2. The preparation according to claim 1, which is an aqueous formulation.

3. The preparation according to claim 2, wherein the aqueous formulation comprises histidine in an amount of from 5 mM to 100 mM, preferably in an amount of from 10 mM to 50 mM.

4. The preparation according to claim 2, wherein the formulation has a pH of from 7.0 to 7.8 or from 7.2 to 7.6.

5. The preparation according to claim 3, wherein the formulation has a pH of from 7.0 to 7.8 or from 7.2 to 7.6.

6. The preparation according to claim 1, further comprising a nonionic surfactant.

7. The preparation according to claim 6, wherein the nonionic surfactant is a polysorbate surfactant such as polysorbate 20 or polysorbate 80.

8. The preparation according to claim 6, wherein the preparation is an aqueous formulation comprising the nonionic surfactant in a concentration of from about 0.001% to about 2% (w/v), preferably from about 0.01% to about 0.5% (w/v)

9. The preparation according to claim 7, wherein the preparation is an aqueous formulation comprising the nonionic surfactant in a concentration of from about 0.001% to about 2% (w/v), preferably from about 0.01% to about 0.5% (w/v).

10. The preparation according to claim 2, further comprising an isotonization agent.

11. The preparation according to claim 1, wherein the carrier protein is keyhole limpet hemocyanin or a subunit thereof.

12. The preparation according to claim 1, wherein the immunoglobulin is an immunoglobulin containing a patient-specific (idiotypic) antigen for B-cell non-Hodgkin lymphoma, preferably said immunoglobulin is immunoglobulin G.

13. The preparation according to claim 2, wherein the protein conjugate is present in a concentration of from 0.1 to 10 mg/ml.

14. A preparation according to claim 1 for use in a method for treatment of the human or animal body by therapy such as for therapy of B-cell non-Hodgkin lymphoma.

15. An aqueous pharmaceutical formulation comprising a protein conjugate comprising an immunoglobulin conjugated to a carrier protein, and histidine as a buffering agent, wherein the formulation has a pH of from 7.2 to 7.6 and wherein the carrier protein is keyhole limpet hemocyanin or a subunit thereof.

16. A set of at least two different protein conjugate preparations, each protein conjugate preparation comprising histidine as a buffering agent and a protein conjugate comprising one or more immunoglobulin moieties conjugated to a carrier protein;
wherein the immunoglobulin moieties of each element of said set of protein conjugate preparation have identical complementarity determining regions (CDRs); and
wherein different protein conjugate preparations differ in that the immunoglobulin moieties of the protein conjugates have different CDRs.

17. A method of treating a patient suffering from B-cell non-Hodgkin lymphoma, comprising administering to the patient an aqueous pharmaceutical formulation, said formulation comprising a protein conjugate comprising an immunoglobulin conjugated to a carrier protein, and histidine as a buffering agent; wherein the complementarity determining regions of the immunoglobulin conjugated to the carrier protein elicits an immune response in the patient against the non-Hodgkin lymphoma B-cells.

18. A set of at least two different immunoglobulin preparations, each immunoglobulin preparation comprising histidine as a buffering agent and an immunoglobulin; wherein the immunoglobulin molecules of each immunoglobulin preparation have identical complementarity determining regions (CDRs); and wherein different immunoglobulin preparations differ in that the immunoglobulins of different preparations have different CDRs.


SEQUENCE LISTING (26 sequences)

SEQ ID NO: 1 - protein, 3408 aa, Megathura crenulata
MLSVRLLIVVLALANAENLVRKSVEHLTQEETLDLQAALRELQMDSSSIGFQKIAAAHGAPASCVHKDTSIACCIHGMPTFPHWHRAYVVHMERALQTKRRTSGLPYWDWTEPITQLPSLAADPVYIDSQGGKAHTNYWYRGNIGFLDKKTNRAADDRLFEKVKPGQHTHLMESVLDPLEQDEFCKFEIQFELPHNAIHYLVGGKHDYSMANLEYTAYDPIFFLHHSNVDRIFAIWQRLQELRNKDPKAMDCAQELLHQKMEPFSWEDNDIPLTNDYDTLNLNGMTPEELKTYLDERSSRARAFASFRLKGFGGSANVFVYVCIPDDNDRNDDHCEKAGDFFVLGGPSEMKWQFYRPYLFDLSDTVHKMGMKLDGHYTVKAELFSVNGTALPDDLLPHPVVVHHPEKGFTDPPVKHHQSANLLVRKNINDLTREEVLNLREAFHKFQEDRSVDGYQATAEYHGLPARCPRPDAKDRYACCVHGMPIFPHWHRLFVTQVEDALVGRGATIGIPYWGLPYWDWTEPMTHIPGLAGNKTYVDSHGASHTNPFHSSVIAFEENAPHTKRQIDQRLFKPATFGHHTDLFNQILYAFEQEDYCDFEVQFEITHNTIHAWTGGSEHFSMSSLHYTAFDPLFYFHHSNVDRLWAVWQALQMRRHKPYRAHCAISLEHMHLKPFAFSSPLNNNEKTHANAMPNKIYDYENVLHYTYEDLTFGGISLENIEKMIHENQQEDRIYAGFLLAGIRTSANVDIFIKTTDSVQHKAGTFAVLGGSKEMKWGFDRVFKFDITHVLKDLDLTADGDFEVTVDITEVDGTKLASSLIPHASVIREHARGKLNRVKFDKVPRSRLIRKNVDRLSPEEMNELRKALALLKEDKSAGGFQQLGAFHGEPKWCPSPEASKKFACCVHGMSVFPHWHRLLTVQSENALRRHGYDGALPYWDWTSPLNHLPELADHEKYVDPEDGVEKHNPWFDGHIDTVDKTTTRSVQNKLFEQPEFGHYTSIAKQVLLALEQDNFCDFEIQYEIAHNYIHALVGGAQPYGMASLRYTAFDPLFYLHHSNTDRIWAIWQALQKYRGKPYNVANCAVTSMREPLQPFGLSANINTDHVTKEHSVPFNVFDYKTNFNYEYDTLEFNGLSISQLNKKLEAIKSQDRFFAGFLLSGFKKSSLVKFNICTDSSNCHPAGEFYLLGDENEMPWAYDRVFKYDITEKLHDLKLHAEDHFYIDYEVFDLKPASLGKDLFKQPSVIHEPRIGHHEGEVYQAEVTSANRIRKNIENLSLGELESLRAAFLEIENDGTYESIAKFHGSPGLCQLNGNPISCCVHGMPTFPHWHRLYVVVVENALLKKGSSVAVPYWDWTKRIEHLPHLISDATYYNSRQHHYETNPFHHGKITHENEITTRDPKDSLFHSDYFYEQVLYALEQDNFCDFEIQLEILHNALHSLLGGKGKYSMSNLDYAAFDPVFFLHHATTDRIWAIWQDLQRFRKRPYREANCAIQLMHTPLQPFDKSDNNDEATKTHATPHDGFEYQNSFGYAYDNLELNHYSIPQLDHMLQERKRHDRVFAGFLLHNIGTSADGHVFVCLPTGEHTKDCSHEAGMFSILGGQTEMSFVFDRLYKLDITKALKKNGVHLQGDFDLEIEITAVNGSHLDSHVIHSPTILFEAGTDSAHTDDGHTEPVMIRKDITQLDKRQQLSLVKALESMKADHSSDGFQAIASFHALPPLCPSPAASKRFACCVHGMATFPQWHRLYTVQFQDSLRKHGAVVGLPYWDWTLPRSELPELLTVSTIHDPETGRDIPNPFIGSKIEFEGENVHTKRDINRDRLFQGSTKTHHNWFIEQALLALEQTNYCDFEVQFEIMHNGVHTWVGGKEPYGIGHLHYASYDPLFYIHHSQTDRIWAIWQSLQRFRGLSGSEANCAVNLMKTPLKPFSFGAPYNLNDHTHDFSKPEDTFDYQKFGYIYDTLEFAGWSIRGIDHIVRNRQEHSRVFAGFLLEGFGTSATVDFQVCRTAGDCEDAGYFTVLGGEKEMPWAFDRLYKYDITETLDKMNLRHDEIFQIEVTITSYDGTVLDSGLIPTPSIIYDPAHHDISSHHLSLNKVRHDLSTLSERDIGSLKYALSSLQADTSADGFAAIASFHGLPAKCNDSHNNEVACCIHGMPTFPHWHRLYTLQFEQALRRHGSSVAVPYWDWTKPIHNIPHLFTDKEYYDVWRNKVMPNPFARGYVPSHDTYTVRDVQEGLFHLTSTGEHSALLNQALLALEQHDYCDFAVQFEVMHNTIHYLVGGPQVYSLSSLHYASYDPIFFIHHSFVDKVWAVWQALQEKRGLPSDRADCAVSLMTQNMRPFHYEINHNQFTKKHAVPNDVFKYELLGYRYDNLEIGGMNLHEIEKEIKDKQHHVRVFAGFLLHGIRTSADVQFQICKTSEDCHHGGQIFVLGGTKEMAWAYNRLFKYDITHALHDAHITPEDVFHPSEPFFIKVSVTAVNGTVLPASILHAPTIIYEPGLDHHEDHHSSSMAGHGVRKEINTLTTAEVDNLKDAMRAVMADHGPNGYQAIAAFHGNPPMCPMPDGKNYSCCTHGMATFPHWHRLYTKQMEDALTAHGARVGLPYWDGTTAFTALPTFVTDEEDNPFHHGHIDYLGVDTTRSPRDKLFNDPERGSESFFYRQVLLALEQTDFCQFEVQFEITHNAIHSWTGGLTPYGMSTLEYTTYDPLFWLHHANTDRIWAIWQALQEYRGLPYDHANCEIQAMKRPLRPFSDPINHNAFTHSNAKPTDVFEYSRFNFQYDNLRFHGMTIKKLEHELEKQKEEDRTFAAFLLHGIKKSADVSFDVCNHDGECHFAGTFAILGGEHEMPWSFDRLFRYDITQVLKQMHLEYDSDFTFHMRIIDTSGKQLPSDLIKMPTVEHSPGGKHHEKHHEDHHEDILVRKNIHSLSHHEAEELRDALYKLQNDESHGGYEHIAGFHGYPNLCPEKGDEKYPCCVHGMSIFPHWHRLHTIQFERALKKHGSHLGIPYWDWTQTISSLPTFFADSGNNNPFFKYHIRSINQDTVRDVNEAIFQQTKFGEFSSIFYLALQALEEDNYCDFEVQYEILHNEVHALIGGAEKYSMSTLEYSAFDPYFMIHHASLDKIWIIWQELQKRRVKPAHAGSCAGDIMHVPLHPFNYESVNNDDFTRENSLPNAVVDSHRFNYKYDNLNLHGHNIEELEEVLRSLRLKSRVFAGFVLSGIRTTAVVKVYIKSGTDSDDEYAGSFVILGGAKEMPWAYERLYRFDITETVHNLNLTDDHVKFRFDLKKYDHTELDASVLPAPIIVRRPNNAVFDIIEIPIGKDVNLPPKVVVKRGTKIMFMSVDEAVTTPMLNLGSYTAMFKCKVPPFSFHAFELGKMYSVESGDYFMTASTTELCNDNNLRIHVHVDDE

SEQ ID NO: 2 - protein, 3421 aa, Megathura crenulata
MWTILALLTATLLFEGAFSVDTVVRKNVDSLSSDEVLALEKALDDLQQDDSNQGYQAIAGYHGVPTMCVDKHEKNVACCLHGMPSFPLWHRLYVVQLERALIRKKATISIPYWDWTSELTHLPELVSHPLFVGTEGGKAHDNSWYRADITFLNKKTSRAVDDRLFEKVQPGHHTRLMEGILDALEQDEFCKFEIQFELAHNAIHYLVGGRHTYSMSHLEYTSYDPLFFLHHSNTDRIFAIWQRLQQLRGKDPNSADCAHNLIHTPMEPFDRDTNPLDLTREHAKPADSFDYGRLGYQYDDLSLNGMSPEELNVYLGERAAKERTFASFILSGFGGSANVVVYVCRPAHDEISDDQCIKAGDFFLLGGPTEMKWGFYRAYHFDVTDSVASIDDDGHGHYYVKSELFSVNGSALSNDILRQPTLVHRPAKGHFDKPPVPVAQANLAVRKNINDLTAEETYSLRKAMERFQNDKSVDGYQATVEFHALPARCPRPDAKDRFACCVHGMATFPHWHRLFVTQVEDALLRRGSTIGLPNWDWTMPMDHLPELATSETYLDPVTGETKNNPFHHAQVAFENGVTSRNPDAKLFMKPTYGDHTYLFDSMIYAFEQEDFCDFEVQYELTHNAIHAWVGGSEKYSMSSLHYTAFDPIFYLHHSNVDRLWAIWQALQIRRGKSYKAHCASSQEREPLKPFAFSSPLNNNEKTYHNSVPTNVYDYVGVLHYRYDDLQFGGMTMSELEEYIHKQTQHDRTFAGFFLSYIGTSASVDIFINREGHDKYKVGSFVVLGGSKEMKWGFDRMYKYEITEALKTLNVAVDDGFSITVEITDVDGSPPSADLIPPPAIIFERADAKDFGHSRKIRKAVDSLTVEEQTSLRRAMADLQDDKTSGGFQQIAAFHGEPKWCPSPEAEKKFACCVHGMAVFPHWHRLLTVQGENALRKHGFTGGLPYWDWTRSMSALPHFVADPTYNDAISSQEEDNPWHHGHIDSVGHDTTRDVRDDLYQSPGFGHYTDIAKQVLLAFEQDDFCDFEVQFEIAHNFIHALVGGNEPYSMSSLRYTTYDPIFFLHRSNTDRLWAIWQALQKYRGKPYNTANCAIASMRKPLQPFGLDSVINPDDETREHSVPFRVFDYKNNFDYEYESLAFNGLSIAQLDRELQRRKSHDRVFAGFLLHEIGQSALVKFYVCKHNVSDCDHYAGEFYILGDEAEMPWRYDRVYKYEITQQLHDLDLHVGDNFFLKYEAFDLNGGSLGGSIFSQPSVIFEPAAGSHQADEYREAVTSASHIRKNIRDLSEGEIESIRSAFLQIQKEGIYENIAKFHGKPGLCEHDGHPVACCVHGMPTFPHWHRLYVLQVENALLERGSAVAVPYWDWTEKADSLPSLINDATYFNSRSQTFDPNPFFRGHIAFENAVTSRDPQPELWDNKDFYENVMLALEQDNFCDFEIQLELIHNALHSRLGGRAKYSLSSLDYTAFDPVFFLHHANVDRIWAIWQDLQRYRKKPYNEADCAVNEMRKPLQPFNNPELNSDSMTLKHNLPQDSFDYQNRFRYQYDNLQFNHFSIQKLDQTIQARKQHDRVFAGFILHNIGTSAVVDIYICVEQGGEQNCKTKAGSFTILGGETEMPFHFDRLYKFDITSALHKLGVPLDGHGFDIKVDVRAVNGSHLDQHILNEPSLLFVPGERKNIYYDGLSQHNLVRKEVSSLTTLEKHFLRKALKNMQADDSPDGYQAIASFHALPPLCPSPSAAHRHACCLHGMATFPQWHRLYTVQFEDSLKRHGSIVGLPYWDWLKPQSALPDLVTQETYEHLFSHKTFPNPFLKANIEFEGEGVTTERDVDAEHLFAKGNLVYNNWFCNQALYALEQENYCDFEIQFEILHNGIHSWVGGSKTHSIGHLHYASYDPLFYIHHSQTDRIWAIWQALQEHRGLSGKEAHCALEQMKDPLKPFSFGSPYNLNKRTQEFSKPEDTFDYHRFGYEYDSLEFVGMSVSSLHNYIKQQQEADRVFAGFLLKGFGQSASVSFDICRPDQSCQEAGYFSVLGGSSEMPWQFDRLYKYDITKTLKDMKLRYDDTFTIKVHIKDIAGAELDSDLIPTPSVLLEEGKHGINVRHVGRNRIRMELSELTERDLASLKSAMRSLQADDGVNGYQAIASFHGLPASCHDDEGHEIACCIHGMPVFPHWHRLYTLQMDMALLSHGSAVAIPYWDWTKPISKLPDLFTSPEYYDPWRDAVVNNPFAKGYIKSEDAYTVRDPQDILYHLQDETGTSVLLDQTLLALEQTDFCDFEVQFEVVHNAIHYLVGGRQVYALSSQHYASYDPAFFIHHSFVDKIWAVWQALQKKRKRPYHKADCALNMMTKPMRPFAHDFNHNGFTKMHAVPNTLFDFQDLFYTYDNLEIAGMNVNQLEAEINRRKSQTRVFAGFLLHGIGRSADVRFWICKTADDCHASGMIFILGGSKEMHWAYDRNFKYDITQALKAQSIHPEDVFDTDAPFFIKVEVHGVNKTALPSSAIPAPTIIYSAGEGHTDDHGSDHIAGSGVRKDVTSLTASEIENLRHALQSVMDDDGPNGFQAIAAYHGSPPMCHMHDGRDVACCTHGMASFPHWHRLFVKQMEDALAAHGAHIGIPYWDWTSAFSHLPALVTDHEHNPFHHGHIAHRNVDTSRSPRDMLFNDPEHGSESFFYRQVLLALEQTDFCQFEVQFEITHNAIHSWTGGHTPYGMSSLEYTAYDPLFYLHHSNTDRIWAIWQALQKYRGFQYNAAHCDIQVLKQPLKPFSESRNPNPVTRANSRAVDSFDYERLNYQYDTLTFHGHSISELDAMLQERKKEERTFAAFLLHGFGASADVSFDVCTPDGHCAFAGTFAVLGGELEMPWSFERLFRYDITKVLKQMNLHYDSEFHFELKIVGTDGTELPSDRIKSPTIEHHGGDHHGGDTSGHDHSERHDGFFRKEVGSLSLDEANDLKNALYKLQNDQGPNGYESIAGYHGYPFLCPEHGEDQYACCVHGMPVFPHWHRLHTIQFERALKEHGSHLGIPYWDWTKSMIALPAFFADSSNSNPFYKYHIMKAGHDTARSPSDLLFNQPQLHGYDYLYYLALSTLEEDNYCDFEVHYEILHNAVHLWLGGTETYSMSSLAFSAYDPVFMILHSGLDRLWIIWQELQKLRKKPYNAAKCAGHMMDEPLHPFNYESANHDSFTRANAKPSTVFDSHKFNYHYDNPDVRGNSIQEISAIIHDLRNQPRVFAGFVLSGIYTSANVKIYLVREGHDDENVGSFVVLGGPKEMPWAYERIFKYDITEVANRLNMHHDDTFNFRLEVQSYTGEMVTHHLPEPLIIYRPAKQEYDVLVIPLGSGHKLPPKVIVKRGTRIMFHPVDDTVNRPVVDLGSHTALYNCVVPPFTYNGYELDHAYSLRDGHYYIAGPTKDLCTSGNVRIHIHIEDE

SEQ ID NO: 3 - protein, 243 aa, artificial
  T038 light chain
MGKQMAALCGFLLVALLWLTPDVASGQSALTQPASVSGSLGQSITITCSGITTDINIYNLVSWYQQHPDKAPKLIIYEVNKRPSGVSDRFSGSKSGNTASLIISGLQAEDEADYHCCAYGPRSSSAVFGGGTHLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 4 - DNA, 729 bp, artificial
  T038 light chain
atggggaagcaaatggccgccctgtgtggctttctcctcgtggcgttgctctggctcacgcccgacgtcgcgtcaggtcagtctgccctgactcaacctgcctccgtgtctgggtctcttggacagtcgatcaccatcacctgcagtggaatcaccactgacataaacatttataaccttgtctcctggtatcaacaacacccagacaaagccccaaaactcataatttatgaagtcaataagcggccctctggggtttctgatcgcttctctggctccaagtctggcaacacggcctccttaataatctctgggctccaggctgaggacgaggctgattatcactgctgtgcatatggacctaggagcagttctgctgttttcggaggaggcacccatctgaccgtcctcggccagcctaaggccaatcctacagttactttgtttccaccctcttctgaggaactacaagctaacaaagcaacactcgtgtgccttatttctgacttctatcctggtgcagttactgttgcttggaaagcagatggatcaccagtaaaggctggtgttgaaactacaaagccctccaagcaaagcaacaataagtatgctgcctcttcatacctgtccttaacacctgagcaatggaagagtcatagaagctacagttgtcaggtaactcacgaaggaagtactgttgaaaagactgtcgctccaacagagtgttca

SEQ ID NO: 5 - protein, 483 aa, artificial [sequence omitted]

SEQ ID NO: 6 - DNA, 1449 bp, artificial
  T038 heavy chain
atggggaagcaaatggccgccctgtgtggctttctcctcgtggcgttgctctggctcacgcccgacgtcgcgtcaggtcaggtgctgttgcaggagtcgggcccaggactggtgaagccttcacagaccctgtcgctcatctgcactgtctctggtggctccatccacagtgatgattaccagtggagttggatccggcagcccccagggcagggcctggagtggattgggaacatctcttacagtggcagtacctactacaacccgtccctcaagagtcgagttaccatgtcgctagacacgtccgagaatcatttctccttgaaagtgacctctgtgactgccgcagacacggccatctattactgtgccagagtcgttacacattgcagtcgtggtgactgtttcttttcccgggtcgacgtctggggcccagggacaacggtcaccgtctcctcagccagcactaaaggaccatcagtatttccccttgcaccttcatctaagagtacttctggtggaactgcagctttgggttgcttagtgaaggattatttccctgagcctgttactgtttcttggaattctggtgcccttacttctggtgttcatactttccctgcagtcttacaaagcagtggtttgtattccctgagcagtgttgtaactgttccatcttcaagtctaggtacccagacctacatctgtaatgtcaaccacaagcccagcaatactaaggtcgataagaaggttgagcccaagtcatgtgataagacacatacttgtccaccttgtccagctccagaattgttgggcggaccatctgtttttttgttcccacccaagcctaaagataccctcatgatatccagaacccctgaagttacatgcgtagttgtagacgttagtcatgaggatcctgaggtcaagtttaattggtatgtggatggagtggaagtgcataatgcaaagacaaagccaagagaggagcagtacaactcaacatacagagtggtatcagtgctgactgttcttcaccaagattggctgaatgggaaagagtataagtgcaaggtgtctaataaggctttaccagctcccatcgaaaagaccattagcaaggctaaaggacaaccaagagagcctcaagtatatacacttcctccatcccgtgatgaattgactaagaaccaggtttctctcacatgtctagtgaaggggttttacccatctgacattgctgttgaatgggaaagcaatggccagcctgaaaataactataagaccacaccacctgttctagacagtgatggatcctttttcctgtactccaagctcacagtagacaaatctagatggcaacaagggaacgtgttttcatgcagtgttatgcatgaagcccttcacaaccactatacacagaagtcactgagtctctcacctggtaag

SEQ ID NO: 7 - protein, 239 aa, artificial
  T069 light chain
MANKHLSLSLFLVLLGLSASLASGVIVMTQSPATLSVSPGESATLSCRASQTIYNKLAWYQMKPGQTLRLLIYGASTRAAGAPAKFSGSGSGTEFTLSISSLQSEDFATYYCQQYSNWPLTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - DNA, 645 bp, artificial
  T069 light chain
gtgatagtgatgacgcagtctccagccaccctgtctgtgtctccaggggaaagtgccaccctctcctgcagggccagtcagactatttacaacaagttagcctggtatcagatgaaacctggccagactctcagactcctcatttatggtgcatcaaccagggccgctggtgccccagccaagttcagtggcagtgggtctgggacagagttcactctcagcatcagcagcctgcagtctgaagattttgcaacttattactgtcagcagtacagtaattggcctctgacgacgttcggccaagggaccaaggtggaaatcaaacgaacagttgctgctcctagtgtttttatttttcccccatccgatgaacaattgaaatctggaactgcatccgtagtatgcttgttgaacaatttctaccctagagaagctaaggttcaatggaaagtcgataatgcactacagtctggtaattcacaagagtctgttactgaacaagactctaaggactctacttacagtctttcttcaactcttaccctatcaaaggcagattacgaaaagcataaggtctatgcttgtgaagttacacatcaaggattgagttcaccagttacaaagagttttaaccgtggtgagtgt

SEQ ID NO: 9 - protein, 476 aa, artificial
  T069 heavy chain
MANKHLSLSLFLVLLGLSASLASGEVQLLESGGGLVLPGGFLRLSCVGSGFTFRNYTMNWVRQAPGKGLEWVSGISGGGDRTYYADSVQGRFSISRDNSRNTLFLHMYSLRAEDTALYYCAKNTTAAGDCCFFDSWGQGALVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 1356 bp, artificial
  T069 heavy chain
gaggtgcagttgttggagtcggggggaggcttggtactgccggggggattcctgagactctcctgtgtaggttctggattcacttttagaaattacaccatgaactgggtccgccaggctccagggaaggggctggagtgggtctcaggtatcagtggtggcggtgataggacatactacgcagactccgtgcagggccgcttcagcatctccagagacaattccaggaacactctgtttttgcacatgtacagcctgagagccgaggacacggccctctattactgtgcgaaaaacacaacggccgctggcgactgttgttttttcgactcgtggggccagggagccctggtcaccgtctcctcagccagcactaaaggaccatcagtatttccccttgcaccttcatctaagagtacttctggtggaactgcagctttgggttgcttagtgaaggattatttccctgagcctgttactgtttcttggaattctggtgcccttacttctggtgttcatactttccctgcagtcttacaaagcagtggtttgtattccctgagcagtgttgtaactgttccatcttcaagtctaggtacccagacctacatctgtaatgtcaaccacaagcccagcaatactaaggtcgataagaaggttgagcccaagtcatgtgataagacacatacttgtccaccttgtccagctccagaattgttgggcggaccatctgtttttttgttcccacccaagcctaaagataccctcatgatatccagaacccctgaagttacatgcgtagttgtagacgttagtcatgaggatcctgaggtcaagtttaattggtatgtggatggagtggaagtgcataatgcaaagacaaagccaagagaggagcagtacaactcaacatacagagtggtatcagtgctgactgttcttcaccaagattggctgaatgggaaagagtataagtgcaaggtgtctaataaggctttaccagctcccatcgaaaagaccattagcaaggctaaaggacaaccaagagagcctcaagtatatacacttcctccatcccgtgatgaattgactaagaaccaggtttctctcacatgtctagtgaaggggttttacccatctgacattgctgttgaatgggaaagcaatggccagcctgaaaataactataagaccacaccacctgttctagacagtgatggatcctttttcctgtactccaagctcacagtagacaaatctagatggcaacaagggaacgtgttttcatgcagtgttatgcatgaagcccttcacaaccactatacacagaagtcactgagtctctcacctggtaag

SEQ ID NO: 11 - protein, 239 aa, artificial
  T096 light chain
MANKHLSLSLFLVLLGLSASLASGEIVMTQSPATLSVSPGERATLSCRASQSVSSNLAWYQQKPGQAPRLLIYGASTRATGIPARFSGSGSGTEFTLTISSLQSEDFAVYYCQQYNNWPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - DNA, 720 bp, artificial
  T096 light chain
atggcgaacaaacacttgtccctctccctcttcctcgtcctccttggcctgtcggccagcttggcctcaggtgaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctccgctcactttcggcggagggaccaaggtggagatcaaacgaacagttgctgctcctagtgtttttatttttcccccatccgatgaacaattgaaatctggaactgcatccgtagtatgcttgttgaacaatttctaccctagagaagctaaggttcaatggaaagtcgataatgcactacagtctggtaattcacaagagtctgttactgaacaagactctaaggactctacttacagtctttcttcaactcttaccctatcaaaggcagattacgaaaagcataaggtctatgcttgtgaagttacacatcaaggattgagttcaccagttacaaagagttttaaccgtggtgagtgttaa

SEQ ID NO: 13 - protein, 476 aa, artificial
  T096 heavy chain
MANKHLSLSLFLVLLGLSASLASGQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGSSVAALRRLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - DNA, 1428 bp, artificial
  T096 heavy chain
atggcgaacaaacacttgtccctctccctcttcctcgtcctccttggcctgtcggccagcttggcctcaggtcaggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaggacacggccgtgtattactgtgcgagagggtcttcggtagctgctctccgacgcctatttgactactggggccagggaaccctggtcaccgtctcctcagccagcactaaaggaccatcagtatttccccttgcaccttcatctaagagtacttctggtggaactgcagctttgggttgcttagtgaaggattatttccctgagcctgttactgtttcttggaattctggtgcccttacttctggtgttcatactttccctgcagtcttacaaagcagtggtttgtattccctgagcagtgttgtaactgttccatcttcaagtctaggtacccagacctacatctgtaatgtcaaccacaagcccagcaatactaaggtcgataagaaggttgagcccaagtcatgtgataagacacatacttgtccaccttgtccagctccagaattgttgggcggaccatctgtttttttgttcccacccaagcctaaagataccctcatgatatccagaacccctgaagttacatgcgtagttgtagacgttagtcatgaggatcctgaggtcaagtttaattggtatgtggatggagtggaagtgcataatgcaaagacaaagccaagagaggagcagtacaactcaacatacagagtggtatcagtgctgactgttcttcaccaagattggctgaatgggaaagagtataagtgcaaggtgtctaataaggctttaccagctcccatcgaaaagaccattagcaaggctaaaggacaaccaagagagcctcaagtatatacacttcctccatcccgtgatgaattgactaagaaccaggtttctctcacatgtctagtgaaggggttttacccatctgacattgctgttgaatgggaaagcaatggccagcctgaaaataactataagaccacaccacctgttctagacagtgatggatcctttttcctgtactccaagctcacagtagacaaatctagatggcaacaagggaacgtgttttcatgcagtgttatgcatgaagcccttcacaaccactatacacagaagtcactgagtctctcacctggtaag

SEQ ID NO: 15 - protein, 243 aa, artificial
  T113 light chain
MANKHLSLSLFLVLLGLSASLASGDVVMTQSPLSLPVTPGEPASMSCRSSQSLLHSNGYKYLDWYLQKPGQSPQLLIFLGSSRASGVPDRFSGSGSGTDFTLKINKVKAEDVGIYYCMQGLQTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - DNA, 657 bp, artificial
  T113 light chain
gatgtcgtgatgactcagtctccactctccctgcccgtcacccctggagagccggcctccatgtcctgtaggtctagtcagagcctcctgcatagcaatggatacaagtatttggattggtacttgcagaagccaggacagtctccacaactcctgatctttttgggttctagtcgggcctccggggtccctgacaggttcagtggcagtggatcaggcacagattttacactaaaaattaacaaagtgaaggctgaggatgttgggatttattactgcatgcaaggtctacaaactccaatcaccttcggccaagggacacgactggagattaaacgaacagttgctgctcctagtgtttttatttttcccccatccgatgaacaattgaaatctggaactgcatccgtagtatgcttgttgaacaatttctaccctagagaagctaaggttcaatggaaagtcgataatgcactacagtctggtaattcacaagagtctgttactgaacaagactctaaggactctacttacagtctttcttcaactcttaccctatcaaaggcagattacgaaaagcataaggtctatgcttgtgaagttacacatcaaggattgagttcaccagttacaaagagttttaaccgtggtgagtgt

SEQ ID NO: 17 - protein, 469 aa, artificial
  T113 heavy chain
MANKHLSLSLFLVLLGLSASLASGEVQLVESGGDLVQPGGSLRLSCATSGFNFLSSNMTWVRQVPGKGLEWVAKIKPDGSEKYYADSVKGRFTISRDNAKKSLYLQMTNLRVEDTSVYYCARGQPFDYWGQGTLVAVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - DNA, 1335 bp, artificial
  T113 heavy chain
gccagcactaaaggaccatcagtatttccccttgcaccttcatctaagagtacttctggtggaactgcagctttgggttgcttagtgaaggattatttccctgagcctgttactgtttcttggaattctggtgcccttacttctggtgttcatactttccctgcagtcttacaaagcagtggtttgtattccctgagcagtgttgtaactgttccatcttcaagtctaggtacccagacctacatctgtaatgtcaaccacaagcccagcaatactaaggtcgataagaaggttgagcccaagtcatgtgataagacacatacttgtccaccttgtccagctccagaattgttgggcggaccatctgtttttttgttcccacccaagcctaaagataccctcatgatatccagaacccctgaagttacatgcgtagttgtagacgttagtcatgaggatcctgaggtcaagtttaattggtatgtggatggagtggaagtgcataatgcaaagacaaagccaagagaggagcagtacaactcaacatacagagtggtatcagtgctgactgttcttcaccaagattggctgaatgggaaagagtataagtgcaaggtgtctaataaggctttaccagctcccatcgaaaagaccattagcaaggctaaaggacaaccaagagagcctcaagtatatacacttcctccatcccgtgatgaattgactaagaaccaggtttctctcacatgtctagtgaaggggttttacccatctgacattgctgttgaatgggaaagcaatggccagcctgaaaataactataagaccacaccacctgttctagacagtgatggatcctttttcctgtactccaagctcacagtagacaaatctagatggcaacaagggaacgtgttttcatgcagtgttatgcatgaagcccttcacaaccactatacacagaagtcactgagtctctcacctggtaaggaagttcagctggtggagtctgggggagacttggtccagcctggggggtccctgagactctcatgtgcaacttctggattcaactttttgagttccaatatgacctgggtccgccaggttccagggaaggggctggagtgggtggccaagataaaaccagatggaagtgagaaatactatgcggactctgtgaagggccgattcaccatctccagagacaacgccaagaagtcactgtatttgcaaatgaccaacctgagagtcgaggacacgtctgtatattactgtgcgagagggcagccctttgactactggggccagggaaccctggtcgccgtctcctca

SEQ ID NO: 19 - protein, 240 aa, artificial
  T117 light chain
MGKQMAALCGFLLVALLWLTPDVASGSYELTQPPSVSVSPGQTARITCSGDALPKKYAYWYQQKSGQAPVLVIYEDSKRPSGIPERFSGSSSGTMATLTISGAQVEDEADYYCYSTDSSGNHYVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 20 - DNA, 720 bp, artificial
  T117 light chain
atggggaagcaaatggccgccctgtgtggctttctcctcgtggcgttgctctggctcacgcccgacgtcgcgtcaggttcctatgagctgacacagccaccctcggtgtcagtgtccccaggacaaacggccaggatcacctgctctggagatgcattgccaaaaaaatatgcttattggtatcagcagaagtcaggccaggcccctgtgctggtcatctatgaggacagcaaacgaccctccgggatccctgagagattctctggctccagctcagggacaatggccaccttgactatcagtggggcccaggtggaggatgaagctgactactactgttactcaacagacagcagtggtaatcattatgtcttcggaactgggaccaaggtcaccgtcctcggccagcctaaggccaatcctacagttactttgtttccaccctcttctgaggaactacaagctaacaaagcaacactcgtgtgccttatttctgacttctatcctggtgcagttactgttgcttggaaagcagatggatcaccagtaaaggctggtgttgaaactacaaagccctccaagcaaagcaacaataagtatgctgcctcttcatacctgtccttaacacctgagcaatggaagagtcatagaagctacagttgtcaggtaactcacgaaggaagtactgttgaaaagactgtcgctccaacagagtgttca

SEQ ID NO: 21 - protein, 470 aa, artificial
  T117 heavy chain
MGKQMAALCGFLLVALLWLTPDVASGHVQLQESGPGLVKPSQTLSLTCTVSGASISSSNYNWTWIRQPPGKGLEWIGNISYSGRTNYNPSLKNRVTLSIDTSKNQFSLNLNSVTAADTAVYYCVGGFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - DNA, 1410 bp, artificial
  T117 heavy chain
atggggaagcaaatggccgccctgtgtggctttctcctcgtggcgttgctctggctcacgcccgacgtcgcgtcaggtcacgtgcagctgcaagagtcgggcccaggactggtgaagccttcacagactttgtccctcacctgcactgtctctggtgcctccatcagcagttctaattataactggacttggatccgccagcccccagggaagggcctggagtggattggcaacatctcttacagtgggcgcaccaactacaacccgtccctcaagaatcgcgtgaccctctcaatagacacgtcgaagaaccagttctccctcaacttgaactctgtgactgccgcagacacagccgtctattattgtgtcgggggattcgacccctggggccagggaaccctggtcaccgtctcctcagccagcactaaaggaccatcagtatttccccttgcaccttcatctaagagtacttctggtggaactgcagctttgggttgcttagtgaaggattatttccctgagcctgttactgtttcttggaattctggtgcccttacttctggtgttcatactttccctgcagtcttacaaagcagtggtttgtattccctgagcagtgttgtaactgttccatcttcaagtctaggtacccagacctacatctgtaatgtcaaccacaagcccagcaatactaaggtcgataagaaggttgagcccaagtcatgtgataagacacatacttgtccaccttgtccagctccagaattgttgggcggaccatctgtttttttgttcccacccaagcctaaagataccctcatgatatccagaacccctgaagttacatgcgtagttgtagacgttagtcatgaggatcctgaggtcaagtttaattggtatgtggatggagtggaagtgcataatgcaaagacaaagccaagagaggagcagtacaactcaacatacagagtggtatcagtgctgactgttcttcaccaagattggctgaatgggaaagagtataagtgcaaggtgtctaataaggctttaccagctcccatcgaaaagaccattagcaaggctaaaggacaaccaagagagcctcaagtatatacacttcctccatcccgtgatgaattgactaagaaccaggtttctctcacatgtctagtgaaggggttttacccatctgacattgctgttgaatgggaaagcaatggccagcctgaaaataactataagaccacaccacctgttctagacagtgatggatcctttttcctgtactccaagctcacagtagacaaatctagatggcaacaagggaacgtgttttcatgcagtgttatgcatgaagcccttcacaaccactatacacagaagtcactgagtctctcacctggtaag

SEQ ID NO: 23 - protein, 242 aa, artificial
  T118 light chain
MGKQMAALCGFLLVALLWLTPDVASGQSVVTQPPSASGTPGQRVSISCSGGTSNIGVNTVNWYQQFPGTAPKLLIYNNDQRPSGVPDRFSGSKSGTSASLAISGLQSADEADYYCATWDDSLNGWVFGGGSKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 24 - DNA, 726 bp, artificial
  T118 light chain
atggggaagcaaatggccgccctgtgtggctttctcctcgtggcgttgctctggctcacgcccgacgtcgcgtcaggtcagtctgtggtgactcagccaccctcagcgtctgggacccccgggcagagggtctccatctcttgttctggaggcacctccaatatcggagttaatactgttaactggtaccagcagttcccagggacggcccccaaactcctcatctataataatgatcagcggccctcaggggtccctgaccgattctctggctccaagtctggcacctcagcctccctggccatcagtgggctccagtctgcggatgaggctgattattactgtgccacatgggatgacagcctgaatggttgggtgttcggcggagggtccaagctgaccgtcctcggccagcctaaggccaatcctacagttactttgtttccaccctcttctgaggaactacaagctaacaaagcaacactcgtgtgccttatttctgacttctatcctggtgcagttactgttgcttggaaagcagatggatcaccagtaaaggctggtgttgaaactacaaagccctccaagcaaagcaacaataagtatgctgcctcttcatacctgtccttaacacctgagcaatggaagagtcatagaagctacagttgtcaggtaactcacgaaggaagtactgttgaaaagactgtcgctccaacagagtgttca

SEQ ID NO: 25 - protein, 485 aa, artificial
  T118 heavy chain
MGKQMAALCGFLLVALLWLTPDVASGQVKLVESGGAVVQPGRSLRLSCLASGFTFGNYGMYWVRQAPGRGLEWVALISYDGSNENYADCVKGRFTISRDNSKNTVFLQMDRLRVEDTAIYFCAKNVSVSEHRTSTAKGGNGMDVWGQGTTVIASSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - DNA, 1455 bp, artificial
  T118 heavy chain
atggggaagcaaatggccgccctgtgtggctttctcctcgtggcgttgctctggctcacgcccgacgtcgcgtcaggtcaggtgaaactggtggagtctgggggagccgtggtccagcctgggaggtccctgagactctcctgtctagcctctggattcactttcggaaattatggcatgtactgggtccgccaggctccaggcagggggctggagtgggtggcacttatttcatatgatggaagtaatgagaactatgcagactgcgtgaagggccgattcaccatctccagagacaattccaagaatacggtgtttctgcaaatggaccgcctgagagttgaggacacggccatatatttctgtgcgaaaaatgtgtcagtcagtgaacatagaacatcaaccgcgaaaggggggaacggaatggacgtctggggccaagggaccacggtgatcgcctcctcagccagcactaaaggaccatcagtatttccccttgcaccttcatctaagagtacttctggtggaactgcagctttgggttgcttagtgaaggattatttccctgagcctgttactgtttcttggaattctggtgcccttacttctggtgttcatactttccctgcagtcttacaaagcagtggtttgtattccctgagcagtgttgtaactgttccatcttcaagtctaggtacccagacctacatctgtaatgtcaaccacaagcccagcaatactaaggtcgataagaaggttgagcccaagtcatgtgataagacacatacttgtccaccttgtccagctccagaattgttgggcggaccatctgtttttttgttcccacccaagcctaaagataccctcatgatatccagaacccctgaagttacatgcgtagttgtagacgttagtcatgaggatcctgaggtcaagtttaattggtatgtggatggagtggaagtgcataatgcaaagacaaagccaagagaggagcagtacaactcaacatacagagtggtatcagtgctgactgttcttcaccaagattggctgaatgggaaagagtataagtgcaaggtgtctaataaggctttaccagctcccatcgaaaagaccattagcaaggctaaaggacaaccaagagagcctcaagtatatacacttcctccatcccgtgatgaattgactaagaaccaggtttctctcacatgtctagtgaaggggttttacccatctgacattgctgttgaatgggaaagcaatggccagcctgaaaataactataagaccacaccacctgttctagacagtgatggatcctttttcctgtactccaagctcacagtagacaaatctagatggcaacaagggaacgtgttttcatgcagtgttatgcatgaagcccttcacaaccactatacacagaagtcactgagtctctcacctggtaag